(19) United States
(12) Patent Application Publication
(10) Pub. No.: US 2010/0317516 A1
(43) Pub. Date: Dec. 16, 2010

(54) Sulfonamide Compounds And Their Use As Fungicide

(75) Inventors: Wassilios Grammenos, Ludwigshafen (DE); Jan Klaas Lohmann, Mannheim (DE); Thomas Grote, Wachenheim (DE); Jochen Dietz, Karlsrhue (DE); Bernd Mueller, Frankenthal (DE); Michael Puhl, Lampertheim (DE); Jens Renner, Bad Duerkheim (DE); Sarah Ulmschneider, Bad Duerkheim (DE); Marianna Vrettou, Mannheim (DE); Joachim Rheinheimer, Ludwigshafen (DE)

Correspondence Address: BRINKS, HOFER, GILSON & LIONE, P.O. BOX 110285, RESEARCH TRIANGLE PARK, NC, 27709, US

(73) Assignee: BASF SE, Ludwigshafen (DE)

(21) Appl. No.: 12/918,513

(22) PCT Filed: Feb. 12, 2009
(86) PCT No.: PCT/EP09/51611

§ 371 Date: Aug. 20, 2010

(30) Foreign Application Priority Data
Feb. 22, 2008 (EP) 08101863.2

(51) Int. Cl. A01N 25/26; C07D 417/14; A01N 43/78; A01P 3/00

(52) U.S. Cl. 504/100; 546/256; 546/269.7; 514/333; 514/342

(57) ABSTRACT

The present invention relates to pyridin-4-ylmethyl-sulfonamides of formula I

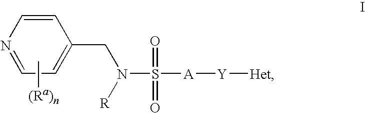

wherein R^{a}, n, R, A, Y and Het are as defined in the claims, and to the N-oxides, and salts thereof and their use for combating harmful fungi, and also to compositions and seed comprising at least one such compound. The invention also relates to a process and intermediates for preparing these compounds.

The present invention relates to novel pyridylmethyl-sulfonamide compounds and the N-oxides, and salts thereof and their use for combating harmful fungi, and also to compositions and seed comprising at least one such compound.

WO 05/033081 describes 4-pyridylmethyl sulfonamide compounds of formula

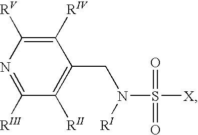

and generally mentions compounds wherein X inter alia may represent an unsubstituted or substituted heteroaryl, and the use of such compounds against plant pathogenic fungi.

WO 06/097489 describes various 4-pyridylmethylamides of biphenylsulfonic acid, wherein the biphenyl moiety may carry substituents at the phenyl ring of the biphenyl moiety at the sulfonamide group. The compounds are used for combating arthropodal pests and for protecting materials against infestation and/or destruction by said pests.

WO 07/104,726 describes specific quinoline methylsulfonamides carrying a biphenyl moiety at the sulfonamide group wherein the phenylene moiety of biphenyl is unsubstituted.

However, with respect to their fungicidal activity, the action of the compounds disclosed is not always completely satisfactory. Based on this, it was an object of the present invention to provide compounds having improved action and/or a broadened activity spectrum against harmful fungi.

This object is achieved by pyridylmethyl-sulfonamide compounds of the general formula I as defined herein and by the N-oxides and their salts, in particular the agriculturally acceptable salts.

Accordingly, the present invention relates to compounds of formula I

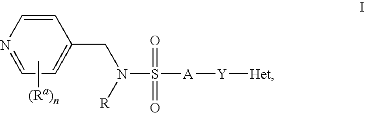

wherein:
R^{a} is halogen, CN, NH₂, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, amino, di(C₁-C₄-alkyl)amino, C₂-C₄-alkenyl, C₂-C₄-haloalkenyl, C₂-C₄-alkynyl, C₂-C₄-haloalkynyl, C₁-C₄-alkoxy-C₁-C₄-alkyl, C₂-C₄-alkenyl, C₂-C₄-alkynyl, C₃-C₈-cycloalkyl or C₁-C₄-alkyl-C₃-C₈-cycloalkyl; and/or
   two radicals R^{a} that are bound to adjacent ring member atoms of the pyridine ring may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and wherein the fused cycle is unsubstituted or carries 1, 2, 3 or 4 identical or different groups as defined for R^{a};
n indicates the number of substituents R^{a} on the pyridine ring and n is 0, 1, 2, 3 or 4, wherein R^{a} radicals are identical or different if n is 2, 3 or 4;
R is hydrogen, C₁-C₄-alkyl, C₂-C₄-alkenyl, C₂-C₄-alkynyl, C₃-C₈-cycloalkyl, C₁-C₄-alkyl-C₃-C₈-cycloalkyl or benzyl wherein the phenyl moiety of benzyl is unsubstituted or carries 1, 2, 3, 4 or 5 substituents selected from the group consisting of cyano, halogen, C₁-C₄-alkyl, C₁-C₄-haloalkyl, C₁-C₄-alkoxy, C₁-C₄-haloalkoxy, C₁-C₄-alkoxycarbonyl and di(C₁-C₄-alkyl)aminocarbonyl;
A is a 5- or 6-membered heteroarenediyl, wherein the ring member atoms of the heteroaryl include besides carbon atoms 1, 2, 3 or 4 heteroatoms selected from the group of N, O and S, and wherein the heteroaryl is unsubstituted or carries 1, 2, 3 or 4 identical or different groups R^{b}:
   R^{b} is halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, C₁-C₄-alkylcarbonyl, C₁-C₄-alkoxycarbonyl, C₁-C₄-alkylamino, di(C₁-C₄-alkyl)amino, C₁-C₄-alkylaminocarbonyl and di(C₁-C₄-alkyl)aminocarbonyl;
Y is a divalent group selected from —O—, —O—CH₂—, —CH₂—O—, —S—, —S(═O)—, —S(═O)₂—, C₁-C₄-alkanediyl, —N(R^{Π})— and —C(NOR^{Π})—,
   R^{Π} is hydrogen or C₁-C₄-alkyl:
Het is a 5- or 6-membered heteroaryl, wherein the ring member atoms of the heteroaryl include besides carbon atoms 1, 2, 3 or 4 heteroatoms selected from the group of N, O and S and wherein the heteroaryl is unsubstituted or carries 1, 2, 3 or 4 identical or different groups R^{c}:
   R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkylthio, C₁-C₆-haloalkylthio, C₁-C₆-alkylsulfinyl, C₁-C₆-haloalkylsulfinyl, C₁-C₆-alkyl-sulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C(═O)R′, C(═NOR″)R′″, C₃-C₈-cycloalkyl, C₁-C₄-alkyl-C₃-C₈-cycloalkyl, phenyl, phenoxy, phenoxy-C₁-C₄-alkyl or a 5- or 6-membered heteroaryl, wherein the ring member atoms of the heteroaryl include besides carbon atoms 1, 2, 3 or 4 hetero-atoms selected from the group of N, O and S, and wherein the aforementioned cyclic radicals are unsubstituted or carry 1, 2, 3 or 4 identical or different substituents R^{d}:
      R′ is hydrogen, NH₂, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkoxy, C₁-C₄-haloalkoxy, C₁-C₄-alkylamino or di(C₁-C₄-alkyl)amino;
      R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl,
      R′″ is hydrogen or C₁-C₄-alkyl;
      R^{d} is halogen, CN, C₁-C₄-alkyl, C₁-C₄-haloalkyl, C₁-C₄-alkoxy or C₁-C₄-haloalkoxy;
   and/or two radicals R^{c} that are bound to adjacent ring member atoms of the Het group may form together with said ring member atoms a fused 5-, 6- or 7-membered saturated, partially unsaturated or aromatic cycle, which may be a carbocycle or heterocycle, wherein the ring member atoms of the fused heterocycle include besides carbon atoms 1, 2, 3 or 4 heteroatoms selected from the group of N, O and S, and wherein the fused cycle is unsubstituted or carries 1, 2, 3 or 4 identical or different radicals R^{e}:
      R^{e} is halogen, CN, C₁-C₄-alkyl, C₁-C₄-haloalkyl, C₁-C₄-alkoxy or C₁-C₄-haloalkoxy;
         and the N-oxides and the agriculturally acceptable salts thereof.

The present invention furthermore relates to processes and intermediates for preparing the compounds of formula I, to agrochemical compositions comprising a solvent or solid carrier and at least one compound of formula I or an N-oxide or an agriculturally acceptable salt thereof, to a method for combating harmful fungi, which process comprises treating the fungi or the materials, plants, the soil or seeds to be protected against fungal attack, with an effective amount of at least one compound of formula I or of an N-oxide or an agriculturally acceptable salt thereof, and to seed comprising a compound of formula I, or an N-oxide or an agriculturally acceptable salt thereof, in an amount of from 0.1 g to 10 kg per 100 kg of seed.

Depending on the substitution pattern, the compounds of formula I and their N-oxides may have one or more centers of chirality, in which case they are present as pure enantiomers or pure diastereomers or as enantiomer or diastereomer mixtures. Both, the pure enantiomers or diastereomers and their mixtures are subject matter of the present invention.

Agriculturally acceptable salts of the compounds I encompass the salts of those cations or the acid addition salts of those acids whose cations and anions, respectively, have no adverse effect on the fungicidal action of the compounds of formula I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound of formula I with an acid of the corresponding anion, preferably of hydrochloric acid, hydrobromic acid, sulfuric acid, phosphoric acid or nitric acid.

The compounds of formula I can be present in atropisomers arising from restricted rotation about a single bond of asymmetric groups. They also form part of the subject matter of the present invention.

In respect of the variables, the embodiments of the intermediates correspond to the embodiments of the compounds of formula I.

The term “compounds I” refers to compounds of formula I. Likewise, this terminology applies to all subformulae herein.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₄-alkyl” refers to a straight-chained or branched saturated hydrocarbon group having 1 to 4 carbon atoms, for example methyl, ethyl, propyl, 1-methylethyl, butyl, 1-methylpropyl, 2-methylpropyl, and 1,1-dimethylethyl. Likewise, the term “C₁-C₆-alkyl” refers to a straight-chained or branched saturated hydrocarbon group having 1 to 6 carbon atoms.

The term “C₁-C₄-haloalkyl” refers to a straight-chained or branched alkyl group having 1 to 4 carbon atoms, wherein some or all of the hydrogen atoms in these groups may be replaced by halogen atoms, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-fluoromethyl-2-fluoroethyl, 1-chloromethyl-2-chloroethyl, 1-bromomethyl-2-bromoethyl, 4-fluorobutyl, 4-chlorobutyl, 4-bromobutyl or nonafluorobutyl. Likewise, the term “C₁-C₆-haloalkyl” refers to a straight-chained or branched alkyl group having 1 to 6 carbon atoms.

The term “C₁-C₄-alkoxy” refers to a straight-chain or branched alkyl group having 1 to 4 carbon atoms which is bonded via an oxygen, at any position in the alkyl group, for example methoxy, ethoxy, n-propoxy, 1-methylethoxy, butoxy, 1-methyl [symbol] propoxy, 2-methylpropoxy or 1,1-dimethylethoxy. Likewise, the term “C₁-C₄-alkoxy” refers to a straight-chain or branched alkyl group having 1 to 6 carbon atoms.

The term “C₁-C₄-haloalkoxy” refers to a C₁-C₄-alkoxy group, wherein some or all of the hydrogen atoms may be replaced by halogen atoms, for example, fluoromethoxy, difluoromethoxy, trifluoromethoxy, dichloromethoxy, trichloromethoxy, chlorofluoromethoxy, dichlorofluoromethoxy, chloro-difluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,2-trifluoroethoxy, pentafluoroethoxy, 1-fluoromethyl-2-fluoroethoxy, 1-chloromethyl-2-chloroethoxy, 1-bromomethyl-2-bromoethoxy, 4-fluorobutoxy, 4-chlorobutoxy, 4-bromobutoxy or nonafluorobutoxy. Likewise, the term “C₁-C₆-haloalkoxy” refers to a C₁-C₆-alkoxy group, wherein some or all of the hydrogen atoms may be replaced by halogen atoms.

The term “C₁-C₄-alkoxy-C₁-C₄-alkyl” refers to alkyl having 1 to 4 carbon atoms, wherein one hydrogen atom of the alkyl radical is replaced by a C₁-C₄-alkoxy group. Likewise, the term “C₁-C₆-alkoxy-C₁-C₄-alkyl” refers to alkyl having 1 to 4 carbon atoms, wherein one hydrogen atom of the alkyl radical is replaced by a C₁-C₆-alkoxy group.

The term “C₁-C₄-haloalkoxy-C₁-C₄-alkyl” refers to alkyl having 1 to 4 carbon atoms, wherein one hydrogen atom of the alkyl radical is replaced by a C₁-C₄-haloalkoxy group. Likewise, the term “C₁-C₆-haloalkoxy-C₁-C₄-alkyl” refers to alkyl having 1 to 4 carbon atoms, wherein one hydrogen atom of the alkyl radical is replaced by a C₁-C₆-alkoxy group.

The term “C₁-C₄-alkoxy-C₁-C₄-alkoxy” refers to a C₁-C₄-alkoxy-C₁-C₄-alkyl group, which is bonded via an oxygen atom to the remainder of the molecule.

The term “C₁-C₄-alkylthio” as used herein refers to straight-chain or branched alkyl groups having 1 to 4 carbon atoms bonded via a sulfur atom, at any position in the alkyl group, for example methylthio, ethylthio, propylthio, isopropylthio, and n-butylthio. Likewise, the term “C₁-C₆-alkylthio” refers to straight-chain or branched alkyl groups having 1 to 6 carbon atoms bonded via a sulfur atom. Accordingly, the terms “C₁-C₄-haloalkylthio” and “C₁-C₆-haloalkylthio” refer to straight-chain or branched haloalkyl groups having 1 to 4 or 1 to 6 carbon atoms bonded through a sulfur atom, at any position in the haloalkyl group.

The terms “C₁-C₄-alkylsulfinyl” or “C₁-C₆-alkylsulfinyl” refer to straight-chain or branched alkyl groups having 1 to 4 or 1 to 6 carbon atoms bonded through a —S(═O)— moiety, at any position in the alkyl group, for example methylsulfinyl and ethylsulfinyl, and the like. Accordingly, the terms “C₁-C₄-haloalkylsulfinyl” and “C₁-C₆-haloalkylsulfinyl”, respectively, refer to straight-chain or branched haloalkyl groups having 1 to 4 and 1 to 6 carbon atoms, respectively, bonded through a —S(═O)— moiety, at any position in the haloalkyl group.

The terms “C₁-C₄-alkylsulfonyl” and “C₁-C₆-alkylsulfonyl”, respectively, refer to straight-chain or branched alkyl groups having 1 to 4 and 1 to 6 carbon atoms, respectively, bonded through a —S(═O)₂— moiety, at any position in the alkyl group, for example methylsulfonyl. Accordingly, the terms “C₁-C₄-haloalkylsulfonyl” and “C₁-C₆-haloalkylsulfonyl”, respectively, refer to straight-chain or branched haloalkyl groups having 1 to 4 and 1 to 6 carbon atoms, respectively, bonded through a —S(═O)₂— moiety, at any position in the haloalkyl group.

The term “C₁-C₄-alkylamino” refers to an amino radical carrying one C₁-C₄-alkyl group as substituent, for example methylamino, ethylamino, propylamino, 1-methylethylamino, butylamino, 1-methylpropylamino, 2-methylpropylamino and 1,1-dimethylethylamino. Likewise, the term “C₁-C₆-alkylamino” refers to an amino radical carrying one C₁-C₆-alkyl group as substituent.

The term “di(C₁-C₄-alkyl)amino” refers to an amino radical carrying two identical or different C₁-C₄-alkyl groups as substituents, for example dimethylamino, diethylamino, di-propylamino, diisopropylamino, N-ethyl-N-methylamino, N-propyl-N-methylamino, N-isopropyl-N-methylamino, N-butyl-N-methylamino, N-pentyl-N-methylamino, N-(1-methylpropyl)-N-methylamino, N-(2-methylpropyl)-N-methylamino, and the like. Likewise, the term “di(C₁-C₆-alkyl)amino” refers to an amino radical carrying two identical or different C₁-C₆-alkyl groups as substituents.

The term “(C₁-C₄-alkoxy)carbonyl” refers to a C₁-C₄-alkoxy radical which is attached via a carbonyl group.

The term “di(C₁-C₄-alkyl)aminocarbonyl” refers to a di(C₁-C₄)alkylamino radical which is attached via a carbonyl group.

The term “phenoxy” and refers to a phenyl radical which is attached via an oxygen atom. Likewise, the term “phenoxy-C₁-C₄-alkyl” and refers to a phenoxy radical which is attached via a C₁-C₄-alkyl group.

The term “C₂-C₄-alkenyl” refers to a straight-chain or branched unsaturated hydrocarbon radical having 2 to 4 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl. Likewise, the term “C₂-C₆-alkenyl” refers to a straight-chain or branched unsaturated hydrocarbon radical having 2 to 6 carbon atoms and a double bond in any position.

The term “C₂-C₄-alkynyl” refers to a straight-chain or branched unsaturated hydrocarbon radical having 2 to 4 carbon atoms and containing at least one triple bond, such as ethynyl, 1-propynyl, 2-propynyl (propargyl), 1-butynyl, 2-butynyl, 3-butynyl, 1-methyl-2-propynyl. Likewise, the term “C₂-C₆-alkynyl” refers to a straight-chain or branched unsaturated hydrocarbon radical having 2 to 6 carbon atoms and at least one triple bond.

The term “C₃-C₈-cycloalkyl” refers to monocyclic saturated hydrocarbon radicals having 3 to 8 carbon ring members, such as cyclopropyl (C₃C₅), cyclobutyl, cyclopentyl, cyclohexyl, cycloheptyl or cyclooctyl.

The term “C₁-C₄-alkyl-C₃-C₈-cycloalkyl” refers to a cycloalkyl radical having 3 to 8 carbon atoms, wherein one hydrogen atom of the cycloalkyl radical is replaced by a C₁-C₄-alkyl group.

The term “5-, 6- or 7-membered carbocycle” is to be understood as meaning both saturated or partially unsaturated carbocycles having 5, 6 or 7 ring members as well as phenyl. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl, and the like.

The term “5-, 6-, or 7-membered heterocycle” wherein the ring member atoms of the heterocycle include besides carbon atoms 1, 2, 3 or 4 heteroatoms selected from the group of N, O and S, is to be understood as meaning both saturated and partially unsaturated as well as aromatic heterocycles having 5, 6 or 7 ring atoms.

Examples include:
saturated and partially unsaturated 5-, 6-, or 7-membered heterocycle wherein the ring member atoms of the heterocycle include besides carbon atoms 1, 2 or 3 heteroatoms selected from the group of N, O and S, and which is saturated or partially unsaturated, for example pyrrolidin-2-yl, pyrrolidin-3-yl, tetrahydrofuran-2-yl, tetrahydrofuran-3-yl, tetrahydrothien-2-yl, tetrahydrothien-3-yl, 1,3-dioxolan-4-yl, isoxazolidin-3-yl, isoxazolidin-4-yl, isoxazolidin-5-yl, isothiazolidin-3-yl, isothiazolidin-4-yl, isothiazolidin-5-yl, pyrazolidin-3-yl, pyrazolidin-4-yl, pyrazolidin-5-yl, oxazolidin-2-yl, oxazolidin-4-yl, oxazolidin-5-yl, thiazolidin-2-yl, thiazolidin-4-yl, thiazolidin-5-yl, imidazolidin-2-yl, imidazolidin-4-yl, 2-pyrrolin-2-yl, 2-pyrrolin-3-yl, 3-pyrrolin-2-yl, 3-pyrrolin-3-yl, piperidin-2-yl, piperidin-3-yl, piperidin-4-yl, 1,3-dioxan-5-yl, tetrahydropyran-2-yl, tetrahydropyran-4-yl, tetrahydrothien-2-yl, hexahydropyridazin-3-yl, hexahydropyridazin-4-yl, hexahydropyrimidin-2-yl, hexahydropyrimidin-4-yl, 5-hexahydropyrimidinyl and piperazin-2-yl;
5-membered heteroaryl (heteroaromatic radical), wherein the ring member atoms of the heteroaryl include besides carbon atoms 1, 2 or 3 heteroatoms selected from the group of N, O and S, for example pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl;
6-membered heteroaryl (heteroaromatic radical), wherein the ring member atoms of the heteroaryl include besides carbon atoms 1, 2 or 3 heteroatoms selected from the group of N, O and S, for example pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl.

The term “C₁-C₄-alkanediyl” refers to a divalent, branched, or straight-chain saturated hydrocarbon radical having 1 to 4 carbon atoms, derived by the removal of one hydrogen atom from each of two different carbon atoms of a parent C₁-C₄-alkane, or by the removal of two hydrogen atoms from a single carbon atom of a parent C₁-C₄-alkane, for example, methanediyl, ethan-1,1-diyl, ethan-1,2-diyl, propan-1,1-diyl, propan-1,2-diyl, propan-2,2-diyl, propan-1,3-diyl, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, and the like.

Furthermore, the term “5- or 6-membered heteroarenediyl” refers to a divalent radical derived from a heteroaryl having two points of attachment. Examples of heteroarenediyl radicals are, for example, divalent radicals derived from pyridine, pyrimidine, pyridazine, 1,2,3-triazine, 1,2,4-triazine, 1,2,3,4-tetrazine, furan, thiophene, pyrrole, thiazole, thiadiazole, pyrazole, imidazole, triazole, tetrazole, oxazole, isoxazole, isothiazole, oxadiazole and the like. The aforementioned groups can be C-attached or N-attached where such is possible. For example, a group derived from pyrrole, imidiazole or pyrazole can be N-attached or C-attached.

The term “two radicals R^{a} that are bound to adjacent ring member atoms of the pyridine ring may form together with said ring member atoms a fused cycle” refers to a condensed bicyclic ring system, wherein the pyridine ring carries a fused-on 5-, 6- or 7-membered carbocyclic or heterocyclic ring.

The term “two radicals R^{c} that are bound to adjacent ring member atoms of the Het group may form together with said ring member atoms a fused cycle” refers to a condensed bicyclic ring system, wherein the 5- or 6-membered heteroaryl, carry a fused-on 5-, 6- or 7-membered carbocyclic or heterocyclic ring.

Preference is given to those compounds I and where applicable also to intermediates and to compounds of all sub-formulae provided herein, for example formula I.1 or formulae I.A to I.J, wherein the substituents and variables (e.g. Het, A, Y, n, R^{a}, R^{b}, R^{c}, R^{d}, R^{e}, R′, R″ and R′″) have independently of each other or more preferably in combination the following meanings:

One embodiment relates to compounds I, wherein n is 0 and the pyridine ring is unsubstituted. Another embodiment relates to compounds I, wherein n is 1 or 2 and the pyridine ring of compounds I carries one or two radicals R^{a}. A further embodiment relates to compounds I, wherein n is 1 and the pyridine ring of compounds I carries one radical R^{a}. A further embodiment relates to compounds I, wherein n is 2 and the pyridine ring of compounds I carries two radicals R^{a}. A further embodiment relates to compounds I, wherein n is 3 and the pyridine ring of compounds I carries three radicals R^{a}.

A further embodiment relates to compounds I, wherein two radicals R^{a} that are bound to adjacent ring member atoms of the pyridine ring do not form together with said ring member atoms any fused cycle.

Preferably, R^{a} is halogen, CN, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₂-C₄-alkynyl, C₁-C₄-alkoxy-C₁-C₄-alkyl, C₃-C₈-cycloalkyl or C₁-C₄-alkyl-C₃-C₈-cycloalkyl. Even more preferably, R^{a} is halogen, CN, C₁-C₄-alkyl, C₁-C₄-haloalkyl, C₁-C₄-alkoxy, C₁-C₄-haloalkoxy, C₁-C₄-alkoxy-C₁-C₄-alkyl, C₃-C₈-cycloalkyl or C₁-C₄-alkyl-C₃-C₈-cycloalkyl.

A further embodiment relates to compounds I, wherein R^{a} is halogen and preferably selected from fluorine and chlorine and in particular, R^{a} is chlorine.

A further embodiment relates to compounds I, wherein R^{a} is CN.

A further embodiment relates to compounds I, wherein R^{a} is C₁-C₄-alkyl and preferably selected from methyl, ethyl, n-propyl and i-propyl.

A further embodiment relates to compounds I, wherein R^{a} is C₁-C₄-haloalkyl, more preferably, R^{a} is C₁-haloalkyl and in particular, R^{a} is trifluoromethyl.

A further embodiment relates to compounds I, wherein R^{a} is C₁-C₄-alkoxy and preferably selected from methoxy, ethoxy, n-propyloxy and i-propyloxy.

A further embodiment relates to compounds I, wherein R^{a} is C₁-C₄-haloalkoxy and preferably halomethoxy such as difluoromethoxy, trifluoromethoxy, dichloromethoxy and trichloromethoxy; haloethoxy such as 2,2-difluoroethoxy, 2,2,2-trifluoroethoxy, 2,2-dichloroethoxy and 2,2,2-trichloroethoxy; halo-n-propoxy, halo-i-propoxy, halo-n-butoxy, halo-1-methyl-propoxy, halo-2-methyl-propoxy or halo-1,1-dimethylethoxy.

A further embodiment relates to compounds I, wherein R^{a} is C₃-C₈-cycloalkyl and preferably selected from cyclopropyl, cylopentyl and cyclohexyl, and in particular, R^{a} is cyclopropyl.

Preference is given to compounds I, wherein R^{a} is selected from F, Cl, Br, OH, SH, CN, C₁-C₂-alkyl, cyclopropyl, CH═CH₂, C≡CH, C₁-C₂-alkoxy, methylthio, methylamino, dimethylamino, CF₃, CHF₂, OCF₃ and OCHF₂.

More preference is given to compounds I, wherein R^{a} is selected from F, Cl, Br, CN, C₁-C₂-alkyl, C₁-C₂-alkoxy, CF₃, CHF₂, OCF₃ and OCHF₂.

A further embodiment relates to compounds I, wherein two radicals R^{a} that are bound to adjacent ring member atoms of the pyridine ring form together with said ring member atoms a fused cycle being a fused 5-, 6- or 7-membered saturated, partially unsaturated or aromatic carbocycle or heterocycle, wherein the ring member atoms of the fused heterocycle include besides carbon atoms 1, 2, 3 or 4 heteroatoms selected from the group of N, O and S, and wherein the fused cycle is unsubstituted and carries 1, 2, 3 or 4 identical or different R^{a} radicals. In one embodiment, the fused cycle is preferably phenyl. In a another embodiment, the fused cycle is preferably a saturated carbocycle and in particular cyclohexyl. In a further embodiment, the fused cycle is preferably a partially unsaturated carbocycle and in particular cyclohexenyl.

Preference is given to compounds I, wherein two radicals R^{a} that are bound to adjacent ring member atoms of the Het group form together with said ring member atoms a fused optionally substituted 6-membered heteroaryl, preferably pyridyl.

Preference is given to compounds I, wherein two radicals R^{a} that are bound to adjacent ring member atoms of the pyridine ring form together with said ring member atoms a fused optionally substituted 5-membered heteroaryl, more preferably furanyl, thienyl or pyrrolyl.

In one embodiment, the two radicals R^{a} that are bound to adjacent ring member atoms of the pyridine ring form together with said ring member atoms a fused cycle, wherein the fused cycle is unsubstituted.

In another embodiment, two radicals R^{a} that are bound to adjacent ring member atoms of the pyridine ring form together with said ring member atoms a fused cycle and wherein the fused cycle is substituted by 1, 2, 3 or 4 identical or different R^{a} radicals selected from halogen, CN, C₁-C₄-alkyl, C₁-C₄-alkoxy, C₁-C₄-haloalkyl and C₁-C₄-haloalkoxy.

One embodiment is related to compounds I, wherein the pyridin-4-yl moiety

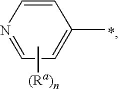

wherein * indicates the point of attachment to the methylene bridge bound to the nitrogen atom of the sulfonamide group, is selected from pyridin-4-yl, 2-methylpyridin-4-yl, 3-methylpryridin-4-yl, 2-ethylpyridin-4-yl, 3-ethylpryridin-4-yl, 2,3-dimethylpyridin-4-yl, 2,3-diethylpyridin-4-yl, 2-methoxypyridin-4-yl, 3-methoxypryrid-4-yl, 2-difluoromethoxypyridin-4-yl, 2-cyanopyridin-4-yl, 2-chloropyridin-4-yl, 2-bromopyridin-4-yl, 2-chloro-3-methylpyridin-4-yl, 3-chloro-2-methylpyridin-4-yl, 2-chloro-3-ethylpyridin-4-yl, 3-chloro-2-ethylpyridin-4-yl, 2-methoxy-3-methylpyridin-4-yl, 3-methoxy-2-methylpyridin-4-yl and 2-methyl-3-ethylpyridin-4-yl and 2-ethyl-3-methyl-pyridin-4-yl.

Particularly preferred embodiments relate to compounds of formula I.1

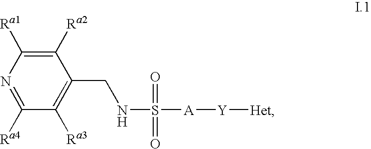

wherein R^{a1}, R^{a2}, R^{a3} and R^{a4} are each independently hydrogen or have one of the definitions specified for R^{a} and wherein the pyridinyl group carries one of the following combinations of the radicals R^{a1}, R^{a2}, R^{a3} and R^{a4} as defined in Table P. below.

TABLE P

| | | | | |
|---|---|---|---|---|
| line | R^{1a} | R^{1b} | R^{1c} | R^{1d} |

| | | | | |
|---|---|---|---|---|
| P-1 | H | H | H | H |
| P-2 | F | H | H | H |
| P-3 | Cl | H | H | H |
| P-4 | Br | H | H | H |
| P-5 | CH₃ | H | H | H |
| P-6 | C₂H₅ | H | H | H |
| P-7 | C₃H₅ | H | H | H |
| P-8 | CF₃ | H | H | H |
| P-9 | CN | H | H | H |
| P-10 | OCH₃ | H | H | H |
| P-11 | OC₂H₅ | H | H | H |
| P-12 | OCF₃ | H | H | H |
| P-13 | OCHF₂ | H | H | H |
| P-14 | C≡CCH₃ | H | H | H |
| P-15 | H | F | H | H |
| P-16 | H | Cl | H | H |
| P-17 | H | Br | H | H |
| P-18 | H | CH₃ | H | H |
| P-19 | H | C₂H₅ | H | H |
| P-20 | H | C₃H₅ | H | H |
| P-21 | H | CF₃ | H | H |
| P-22 | H | CN | H | H |
| P-23 | H | OCH₃ | H | H |
| P-24 | H | OC₂H₅ | H | H |
| P-25 | H | OCF₃ | H | H |
| P-26 | H | OCHF₂ | H | H |
| P-27 | H | C≡CH₃ | H | H |
| P-28 | CH₃ | CH₃ | H | H |
| P-29 | C₂H₅ | CH₃ | H | H |
| P-30 | OCH₃ | CH₃ | H | H |
| P-31 | CH₃ | C₂H₅ | H | H |
| P-32 | C₂H₅ | C₂H₅ | H | H |
| P-33 | OCH₃ | C₂H₅ | H | H |
| P-34 | CH₃ | OCH₃ | H | H |
| P-35 | C₂H₅ | OCH₃ | H | H |
| P-36 | OCH₃ | OCH₃ | H | H |

| | | | |
|---|---|---|---|
| P-37 | %-(CH)₄-# | H | H |

wherein “%” indicates the point of attachment to the pyridine ring at the position of the R^{a1} substituent; and
“#” indicates the point of attachment to the pyridine ring at the position of the R^{a2} substituent.

Preference is given to compounds I, wherein R is hydrogen, C₁-C₂-alkyl, —CH═CH₂ or —CH₂—C≡CH.

Particular preference is given to compounds I, wherein R is hydrogen.

One embodiment relates to compounds I, wherein A is a heteroarenediyl selected from the group consisting of pyridindiyl, pyrimidindiyl, pyridazindiyl, pyrazindiyl, triazindiyl, furandiyl, thiendiyl, pyrroldiyl, pyrazoldiyl, isoxazoldiyl, isothiazoldiyl, imidazoldiyl, oxazoldiyl, thiazoldiyl, triazoldiyl, thiadiazoldiyl and oxadiazoldiyl, and wherein the 17 aforementioned radicals are unsubstituted or carry 1, 2 or 3 identical or different substituents R^{b}. If one point of attachment is located on a nitrogen atom of the heteroarenediyl radical, said nitrogen atom is attached either to the sulfur atom of the sulfonamide group or to Y, with the point of attachment to Y being more preferred. In one embodiment, A is pyridindiyl. In the another embodiment, A is pyrimidindiyl. In a further embodiment, A is pyridazindiyl. In a further embodiment, A is pyrazindiyl. In a further embodiment, A is furandiyl. In a further embodiment, A is thiendiyl. In a further embodiment, A is pyrroldiyl. In a further embodiment, A is pyrazoldiyl. In a further embodiment, A is isoxazoldiyl. In a further embodiment, A is isothiazoldiyl. In a further embodiment, A is imidazoldiyl. In a further embodiment, A is oxazoldiyl. In a further embodiment, A is thiazoldiyl. In a further embodiment, A is 1,2,4-triazoldiyl. In a further embodiment, A is 1,2,4-thiadiazoldiyl. In a further embodiment, A is 1,2,4-oxadiazoldiyl.

Amongst compounds I, wherein A is a 6-membered heteroarenediyl, particular preference given to those, wherein A is pyridindiyl or pyrimidinyl, wherein each of the aforementioned two radicals are unsubstituted or carry 1, 2 or 3 identical or different substituents R^{b}.

Amongst compounds I, wherein A is a 6-membered heteroarenediyl, most preference is given to those, wherein A is selected from the group consisting of pyridin-2,5-diyl, pyridin-2,6-diyl, pyridin-2,4-diyl, pyridin-3,5-diyl, pyrimidin-2,5-diyl, pyrimidin-2,4-diyl and pyrimidin-4,6-diyl wherein the aforementioned heteroarenediyl radicals are unsubstituted or carry 1, 2, 3 or 4 identical or different substituents R^{b}.

Amongst compounds I, wherein A is a 5-membered heteroarenediyl, particular preference given to those, wherein A is thiendiyl, thiazoldiyl, oxazoldiyl, pyrazoldiyl or pyridindiyl, wherein each of the aforementioned five radicals are unsubstituted or carry 1, 2 or 3 identical or different substituents R^{b}.

Amongst compounds I, wherein A is a 5-membered heteroarenediyl, most preference is given to those, wherein A is selected from the group consisting of thiophen-2,5-diyl, thiophen-2,4-diyl, thiophen-3,5-diyl, thiazol-2,5-diyl, thiazol-2,4-diyl, oxazol-2,5-diyl, oxazol-2,4-diyl, pyrazol-3,5-diyl, pyrazol-1,3-diyl and pyrazol-1,4-diyl, wherein the aforementioned heteroarenediyl radicals are unsubstituted or carry 1, 2, 3 or 4 identical or different substituents R^{b}.

Particularly preferred embodiments relate to compounds I, wherein A is one of the following radicals A-1 to A-54:

| No. | A |
|---|---|
| A-1 | 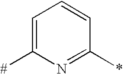 |
| A-2 | 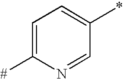 |
| A-3 | 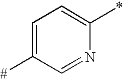 |
| A-4 | 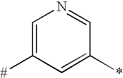 |
| A-5 | 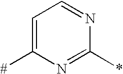 |
| A-6 | 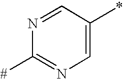 |
| A-7 | 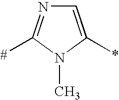 |
| A-8 | 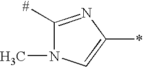 |
| A-9 | 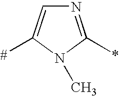 |
| A-10 | 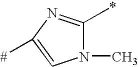 |
| A-11 | 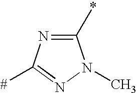 |
| A-12 | 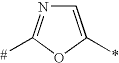 |
| A-13 | 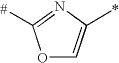 |
| A-14 | 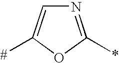 |
| A-15 | 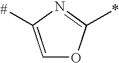 |
| A-16 | 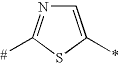 |
| A-17 | 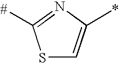 |
| A-18 | 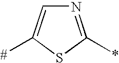 |
| A-19 | 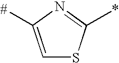 |
| A-20 | 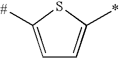 |

wherein “#” indicates the point of attachment to the sulfur atom of the sufonamide group; and “*” indicates the point of attachment to Y.

One embodiment relates to compounds I, wherein the group A carries one or two radicals R^{b}. In another embodiment, the group A is unsubstituted or carries one radical R^{b}. In a further embodiment, the group A is unsubstituted. In a further embodiment, the group A carries 1 radical R^{b}. In a further embodiment, the group A carries 2 radicals R^{b}.

If R^{b} is present, R^{b} is preferably halogen, CN, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, C₁-C₄-alkylcarbonyl, C₁-C₄-alkoxycarbonyl, C₁-C₄-alkylamino, di(C₁-C₄-alkyl)amino, C₁-C₄-alkylaminocarbonyl or di(C₁-C₄-alkyl)aminocarbonyl. More preferably, R^{b} is halogen, CN, C₁-C₄-alkyl, C₁-C₄-haloalkyl, C₁-C₄-alkoxy or C₁-C₄-haloalkoxy.

In a further embodiment, R^{b} is halogen and preferably selected from fluorine and chlorine, and in particular, chlorine. In a further embodiment, R^{b} is CN.

In a further embodiment, R^{b} is C₁-C₄-alkyl and preferably selected from methyl, ethyl, n-propyl and i-propyl, and in particular, methyl.

In a further embodiment, R^{b} is C₁-C₄-haloalkyl. More preferably, R^{b} is C₁-haloalkyl and selected from fluoromethyl, difluoromethyl, trifluoromethyl, chloromethyl, dichloromethyl and trichloromethyl, and in particular, trifluoromethyl.

One embodiment relates to compounds I, wherein Y is —O—, —S— or —NH—.

Another embodiment relates to compounds I, wherein Y is —O—. A more specific embodiment relates to compounds I, wherein R is hydrogen, Y is —O— and R^{a1}, R^{a2}, R^{a3} and R^{a4} are each independently hydrogen or have one of the definitions specified for R^{a}, which compounds are of formula I.A:

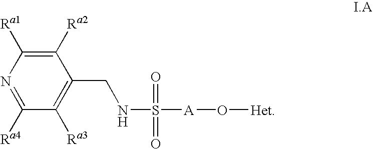

A further embodiment relates to compounds I, wherein Y is —N(R^{Π})—, wherein R^{Π} is hydrogen or C₁-C₄-alkyl. In one embodiment, R^{Π} is C₁-C₄-alkyl, and preferably selected from methyl, ethyl, n-propyl and i-propyl, and in particular, R^{Π} is methyl. In another embodiment, R^{Π} is hydrogen.

A further embodiment relates to compounds I, wherein Y is —N(CH₃)—. A more specific embodiment relates to compounds I, wherein R is hydrogen, Y is —N(CH₃)— and R^{a1}, R^{a2}, R^{a3} and R^{a4} are each independently hydrogen or have one of the definitions specified for R^{a}, which compounds are of formula I.B:

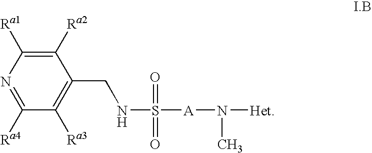

A further embodiment relates to compounds I, wherein Y is —S—. A more specific embodiment relates to compounds I, wherein R is hydrogen, Y is —S— and R^{a1}, R^{a2}, R^{a3} and R^{a4} are each independently hydrogen or have one of the definitions specified for R^{a}, which compounds are of formula I.C

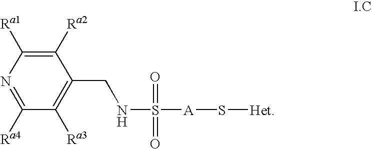

A further embodiment relates to compounds I, wherein Y is —CH₂—. A more specific embodiment relates to compounds I, wherein R is hydrogen, Y is —CH₂— and R^{a1}, R^{a2}, R^{a3} and R^{a4} are each independently hydrogen or have one of the definitions specified for R^{a}, which compounds are of formula I.F:

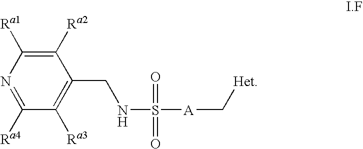

A further embodiment relates to compounds I, wherein Y is —O(CH₂)—. A more specific embodiment relates to compounds I, wherein R is hydrogen, Y is —O(CH₂)— and R^{a1}, R^{a2}, R^{a3} and R^{a4} are each independently hydrogen or have one of the definitions specified for R^{a}, which compounds are of formula I.G:

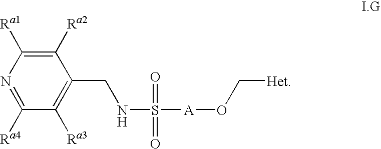

A further embodiment relates to compounds I, wherein Y is —(CH₂)O—. A more specific embodiment relates to compounds I, wherein R is hydrogen, Y is —(CH₂)O— and R^{a1}, R^{a2}, R^{a3} and R^{a4} are each independently hydrogen or have one of the definitions specified for R^{a}, which compounds are of formula I.H:

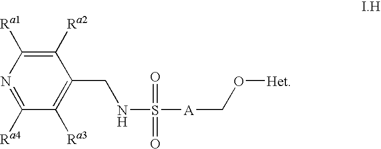

One embodiment relates to compounds I, wherein Het is selected from pyridinyl, pyrimidinyl, pyridazinyl, pyrazinyl, thiazolyl, oxazolyl, isothiazolyl, isoxazolyl, thienyl, furyl, 1,3,5-triazinyl, 1,2,4-triazinyl, thiadiazolyl, 1,2,3-triazolyl, 1,2,4-triazolyl, pyrazolyl, imidazolyl, wherein the aforementioned heteroaryl radicals are unsubstituted or carry 1, 2, 3 or 4 identical or different substituents R^{c}. More preferably, Het is pyrimidin-2-yl, pyrimidin-3-yl, pyrimidin-4-yl, pyridin-3-yl, thiazol-2-yl, pyrazin-2-yl, pyridazin-3-yl, 1,3,5-triazin-2-yl, and 1,2,4-triazin-3-yl, wherein the aforementioned heteroaryl radicals are unsubstituted or carry 1, 2, 3 or 4 identical or different substituents R^{c}.

Another embodiment relates to compounds I, wherein Het is a 6-membered heteroaryl, wherein the ring member atoms of the heteroaryl include besides carbon atoms 1, 2, 3 or 4 heteroatoms selected from the group of N, O and S, and wherein the 6-membered heteroaryl is unsubstituted or carries 1, 2, 3 or 4 identical or different groups R^{c}.

If Het is a 6-membered heteroaryl, Het carries at least one nitrogen as ring member atom. Preference is given to compounds I, wherein Het is a pyridyl radical selected from pyridin-2-yl, pyridin-3-yl and pyridin-4-yl, wherein the aforementioned pyridyl radicals are unsubstituted or carry 1, 2, 3 or 4 identical or different substituents R^{c}.

In another embodiment, Het is pyridin-2-yl, which is unsubstituted or carries 1 or 2 radicals R^{c}. In a more preferred embodiment, Het is selected from 3-trifluoromethylpyridin-2-yl, 5-trifluoromethylpyridin-2-yl, 4-trifluoromethylpyridin-2-yl, 3-chloropyridin-2-yl, 5-chloropyridin-2-yl, 4-chloropyridin-2-yl, 3-bromopyridin-2-yl, 5-bromopyridin-2-yl, 4-bromopyridin-2-yl, 3-trichloromethylpyridin-2-yl, 5-trichloromethylpyridin-2-yl, 4-trichloromethylpyridin-2-yl, 3-cyanopyridin-2-yl, 5-cyanopyridin-2-yl, 4-cyanopyridin-2-yl, 3-nitropyridin-2-yl, 5-nitropyridin-2-yl, 4-nitropyridin-2-yl, 3-methylsulfonylpyridin-2-yl, 5-methylsulfonylpyridin-2-yl, 4-methylsulfonylpyridin-2-yl, 3-ethylsulfonylpyridin-2-yl, 5-ethylsulfonylpyridin-2-yl, 4-ethylsulfonylpyridin-2-yl, 3-methoxycarbonylpyridin-2-yl, 5-methoxycarbonylpyridin-2-yl, 4-methoxycarbonylpyridin-2-yl, 5-aminocarbonylpyridin-2-yl, 4-aminocarbonylpyridin-2-yl, 3-aminocarbonylpyridin-2-yl, 5-N-methylaminocarbonylpyridin-2-yl, 4-N-methylaminocarbonylpyridin-2-yl, 3-N-methylaminocarbonylpyridin-2-yl, 3-methoxypyridin-2-yl, 3-ethoxypyridin-2-yl, 3-difluoromethoxypyridin-2-yl, 5-methoxypyridin-2-yl, 5-ethoxypyridin-2-yl, 5-difluoromethoxypyridin-2-yl, 3-chloro-5-trifluoromethylpyridin-2-yl, 3-fluoro-5-trifluoromethylpyridin-2-yl, 3-bromo-5-trifluoromethylpyridin-2-yl, 3-methyl-5-trifluoromethylpyridin-2-yl, 3-ethyl-5-trifluoromethylpyridin-2-yl, 3-chloro-5-difluoromethoxypyridin-2-yl, 3-fluoro-5-difluoromethoxypyridin-2-yl, 3-methyl-5-difluoromethoxypyridin-2-yl, 3-chloro-5-trichloromethylpyridin-2-yl, 3-fluoro-5-trichloromethylpyridin-2-yl, 3-chloro-5-cyanopyridin-2-yl, 3-fluoro-5-cyanopyridin-2-yl, 3-methyl-5-cyanopyridin-2-yl, 3-ethyl-5-cyanopyridin-2-yl, 3-chloro-5-nitropyridin-2-yl, 3-chloro-5-methoxycarbonylpyridin-2-yl, 3-chloro-5-aminocarbonylpyridin-2-yl, 3-chloro-5-methylaminocarbonylpyridin-2-yl, 3-fluoro-5-nitropyridin-2-yl, 3-fluoro-5-methoxycarbonylpyridin-2-yl, 3-fluoro-5-aminocarbonylpyridin-2-yl, 3-fluoro-5-methylaminocarbonylpyridin-2-yl, 4-chloro-5-trifluoromethylpyridin-2-yl, 4-fluoro-5-trifluoromethylpyridin-2-yl, 4-bromo-5-trifluoromethylpyridin-2-yl, 4-methyl-5-trifluoromethylpyridin-2-yl, 4-chloro-5-nitropyridin-2-yl, 4-chloro-5-cyanopyridin-2-yl, 3-chloro-6-trifluoromethylpyridin-2-yl, 3-fluoro-6-trifluoromethylpyridin-2-yl, 3-methyl-6-trifluoromethylpyridin-2-yl, 4-chloro-5-difluoromethoxypyridin-2-yl, 4-fluoro-5-difluoromethoxypyridin-2-yl, 3-chloro-5-bromopyridin-2-yl, 3,5-dichloropyridin-2-yl, 3,5-difluoropyridin-2-yl, 3,5-dibromopyridin-2-yl, 3-methyl-5-chloropyridin-2-yl, 3-methyl-5-fluoropyridin-2-yl, 3-methyl-5-bromopyridin-2-yl, 3-methoxy-5-trifluoromethylpyridin-2-yl, 3-methoxy-5-cyanopyridin-2-yl, 3-methoxy-5-nitropyridin-2-yl, 3-methoxy-5-difluoromethoxypyridin-2-yl, 3-ethoxy-5-trifluoromethylpyridin-2-yl, 3-ethoxy-5-cyanopyridin-2-yl, 3-ethoxy-5-nitropyridin-2-yl, 3-ethoxy-5-difluoromethoxypyridin-2-yl, 3-chloro-4-methyl-5-trifluoromethylpyridin-2-yl and 3,4-dichloro-5-trifluoromethylpyridin-2-yl.

In a further embodiment, Het is pyridin-3-yl, which is unsubstituted or carries 1 or 2 radicals R^{c}. In a more preferred embodiment, Het is selected from 6-trifluoromethylpyridin-3-yl, 2-trifluoromethylpyridin-3-yl, 4-trifluoromethylpyridin-3-yl, 4-chloro-6-trifluoromethylpyridin-3-yl, 2-chloro-6-trifluoromethylpyridin-3-yl, 2-chloro-5-trifluoro-methylpyridin-3-yl, 4-fluoro-6-trifluoromethylpyridin-3-yl, 4,6-di(trifluoromethyl)pyridin-3-yl, 4,6-dichloropyridin-3-yl, 4-methyl-6-chloropyridin-3-yl, 5-cyanopyridin-3-yl, 5-fluoro-6-cyanopyridin-3-yl, 4-fluoro-6-cyanopyridin-3-yl, 6-methylsulfonylpyridin-3-yl, 5-chloro-6-methylsulfonylpyridin-3-yl and 5-methyl-6-methylsulfonylpyridin-3-yl.

In a further embodiment, Het is a pyridazinyl radical selected from pyridazin-3-yl and pyridazin-4-yl, wherein the aforementioned pyridazinyl radicals are unsubstituted or carry 1, 2 or 3 identical or different substituents R^{c}. More preferably, Het is pyridazin-3-yl, which is unsubstituted or carries 1 or 2 radicals R^{c}. In a particularly preferred embodiment, Het is selected from 4-trifluoromethylpyridazin-3-yl, 4-methyl-6-trifluoromethylpyridazin-3-yl, 4-chloro-6-difluoromethoxypyridazin-3-yl, 4-fluoro-6-difluoromethoxypyridazin-3-yl and 4-methyl-6-difluoromethoxypyridazin-3-yl.

In a further embodiment, Het is a pyrimidinyl radical selected from pyrimidin-2-yl, pyrimidin-4-yl, pyrimidin-5-yl and pyrimidin-6-yl, wherein the aforementioned pyrimidinyl radicals are unsubstituted or carry 1, 2 or 3 identical or different substituents R^{c}. More preferably, Het is pyrimidinyl, which is unsubstituted or carries 1 or 2 radicals R^{c}. In a particularly preferred embodiment, Het is selected from pyrimidin-2-yl, 4-trifluoromethylpyrimidin-2-yl, 5-trifluoromethylpyrimidin-2-yl, 2-trifluoromethylpyrimidin-4-yl, 2-trifluoromethylpyrimidin-5-yl, 4-trifluoromethylpyrimidin-6-yl, 4-cyanopyrimidin-2-yl, 5-cyanopyrimidin-2-yl, 4-(1,1,1-trifluoroethoxy)pyrimidin-2-yl, 5-chloro-6-trifluoromethylpyrimidin-4-yl, 5-fluoro-6-trifluoromethylpyrimidin-4-yl and 5-chloro-2-trifluoromethylpyrimidin-4-yl.

In a further embodiment, Het is a pyrazinyl radical selected from pyrazin-2-yl and pyrazin-3-yl, wherein the aforementioned pyrazinyl radicals are unsubstituted or carry 1, 2 or 3 identical or different substituents R^{c}. More preferably, Het is pyrazin-2-yl, which is unsubstituted or carries 1 or 2 radicals R^{c}. In a particularly preferred embodiment, Het is selected from 6-trifluoromethylpyrazin-2-yl, 5-trifluoromethylpyrazin-2-yl, 3-trifluoromethylpyrazin-2-yl, 3-chloro-5-trifluoromethylpyrazin-2-yl, 3-fluoro-5-trifluoromethylpyrazin-2-yl, 5-chloro-6-trifluoromethylpyrazin-2-yl, 6-trifluoromethylpyridazin-3-yl and 5-trifluoromethylpyridazin-3-yl.

In a further embodiment, Het is a triazinyl radical selected from 1,2,4-triazin-2-yl and 1,2,4-triazin-3-yl, wherein the aforementioned pyrazinyl radicals are unsubstituted or carry 1, 2 or 3 identical or different substituents R^{c}. In a particularly preferred embodiment, Het is selected from 1,2,4-triazin-3-yl, 6-trifluoromethyl-1,2,4-triazin-3-yl, 5-trifluoromethyl-1,2,4-triazin-3-yl, 4,6-bis(trifluoromethyl)-1,3,5-triazin-2-yl, 4,6-bis(difluoromethoxy)-1,3,5-triazin-2-yl and 4,6-bismethoxy-1,3,5-triazin-2-yl.

Another embodiment relates to compounds I, wherein Het is a 5-membered heteroaryl, wherein the ring member atoms of the heteroaryl include besides carbon atoms 1, 2, 3 or 4 heteroatoms selected from the group of N, O and S, and wherein the heteroaryl is unsubstituted or carries 1, 2, 3 or 4 identical or different groups R^{c}.

If Het is a 5-membered heteroaryl, Het carries one heteroatom as ring member atom. In another embodiment, Het is a furanyl radical selected from furan-2-yl and furan-3-yl, wherein the aforementioned furanyl radicals are unsubstituted or carry 1, 2 or 3 identical or different substituents R^{c}. In a further embodiment, Het is a thienyl radical selected from thien-2-yl and thien-3-yl, wherein the aforementioned thienyl radicals are unsubstituted or carry 1, 2 or 3 identical or different substituents R^{c}. In a further embodiment, Het is a pyrrolyl radical selected from pyrrol-2-yl and pyrrol-3-yl, wherein the aforementioned pyrrolyl radicals are unsubstituted or carry 1, 2, 3 or 4 identical or different substituents R^{c}.

If Het is a 5-membered heteroaryl, Het carries two heteroatoms as ring member atoms. In a more preferred embodiment, Het carries at least one nitrogen as ring member atom. In another embodiment, Het is a pyrazolyl radical selected from pyrazol-3-yl, pyrazol-4-yl and pyrazol-5-yl, wherein the aforementioned pyrazolyl radicals are un-substituted or carry 1, 2 or 3 identical or different substituents R^{c}. In a further embodiment, Het is an isoxazolyl radical selected from isoxazol-3-yl, isoxazol-4-yl and isoxazol-5-yl, wherein the aforementioned isoxazolyl radicals are unsubstituted or carry one or two identical or different substituents R^{c}. In a further embodiment, Het is an isothiazolyl radical selected from isothiazol-3-yl, isothiazol-4-yl and isothiazol-5-yl, wherein the aforementioned isothiazolyl radicals are unsubstituted or carry one or two identical or different substituents R^{c}. In a further embodiment, Het is an imidazolyl radical selected from imidazol-2-yl, imidazol-4-yl and imidazol-5-yl, wherein the aforementioned imidazolyl radicals are unsubstituted or carry 1, 2 or 3 identical or different substituents R^{c}. In a further embodiment, Het is an oxazolyl radical selected from oxazol-2-yl, oxazol-4-yl and oxazol-5-yl, wherein the aforementioned oxazolyl radicals are unsubstituted or carry one or two identical or different substituents R^{c}. In a further embodiment, Het is a thiazolyl radical selected from thiazol-2-yl, thiazol-4-yl and thiazol-5-yl, wherein the aforementioned thiazolyl radicals are unsubstituted or carry one or two identical or different substituents R^{c}. More preferably, Het is thiazol-2-yl, which is unsubstituted or carries 1 or 2 radicals R^{c}. In a particularly preferred embodiment, Het is selected from thiazol-2-yl, 5-trifluoromethylthiazol-2-yl and 4-trifluoromethylthiazol-2-yl.

In another preferred embodiment Het is a 5-membered heteroaryl with three heteroatoms as ring member atoms. and more preferably, Het has at least two nitrogens as ring member atoms. In a further embodiment, Het is a 1,2,3-triazolyl radical selected from 1,2,3-triazol-4-yl and 1,2,3-triazol-5-yl, wherein the aforementioned 1,2,3-triazolyl radicals are unsubstituted or carry one or two identical or different substituents R^{c}. In a further embodiment, Het is a 1,2,4-triazolyl radical selected from 1,2,4-triazol-3-yl and 1,2,4-triazol-5-yl, wherein the aforementioned 1,2,4-triazolyl radicals are unsubstituted or carry one or two identical or different substituents R^{c}. In a further embodiment, Het is an 1,2,4-oxadiazolyl radical selected from 1,2,4-oxadiazol-3-yl and 1,2,4-oxadiazol-5-yl, wherein the aforementioned 1,2,4-oxadiazolyl radicals are unsubstituted or carry one substituent R^{c}. In a further embodiment, Het is an 1,3,4-oxadiazolyl radical selected from 1,3,4-oxadiazol-2-yl and 1,3,4-oxadiazol-5-yl, wherein the aforementioned 1,3,4-oxadiazolyl radicals are unsubstituted or carry one substituent R^{c}. In a further embodiment, Het is a 1,2,3-thiadiazolyl radical selected from 1,2,3-thiadiazol-4-yl and 1,2,3-thiadiazol-5-yl, wherein the aforementioned 1,2,3-thiadiazolyl radicals are unsubstituted or carry one substituent R^{c}. In a further embodiment, Het is a 1,2,4-thiadiazolyl radical selected from 1,2,4-thiadiazol-3-yl and 1,2,4-thiadiazol-5-yl, wherein the aforementioned 1,2,4-thiadiazolyl radicals are unsubstituted or carry one substituent R^{c}. In a further embodiment, Het is a 1,3,4-thiadiazolyl radical selected from 1,3,4-thiadiazol-2-yl and 1,3,4-thiadiazol-5-yl, wherein the aforementioned 1,3,4-thiadiazolyl radicals are unsubstituted or carry 1 substituent R^{c}.

Particularly preferred embodiments relate to compounds I, wherein the group Het is one of the following radicals H-1 to H-48:

| No. | Het |
|---|---|
| H-1 | 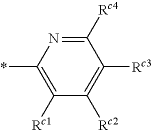 |
| H-2 | 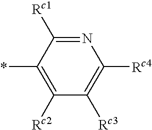 |
| H-3 | 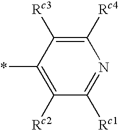 |
| H-4 | 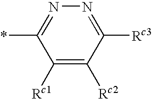 |
| H-5 | 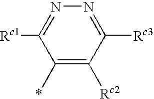 |
| H-6 | 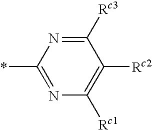 |
| H-7 | 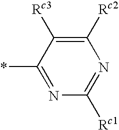 |
| H-8 | 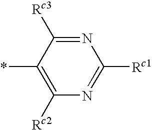 |
| H-9 | 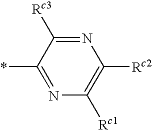 |

wherein “*” indicates the point of attachment to Y; and R^{c1}, R^{c2}, R^{c3} and R^{c4} are each independently hydrogen or have one of the definitions specified for R^{c}, especially those being preferred.

One embodiment relates to compounds I, wherein Het carries 1, 2 or 3 radicals R^{c}. Another embodiment relates to compounds I, wherein Het carries 1 or 2 radicals R^{c}. A further embodiment relates to compounds I, wherein Het carries 1 radical R^{c}. A further embodiment relates to compounds I, wherein Het carries 2 radicals R^{c}. A further embodiment relates to compounds I, wherein Het is unsubstituted.

A further embodiment relates to compounds I, wherein Het carries 1, 2, 3 or 4 four radicals R^{c} which are selected from F, Cl, Br, CN, C₁-C₂-alkylsulfonyl, C₁-C₂-alkoxycarbonyl, aminocarbonyl, C₁-C₂-alkylaminocarbonyl, di(C₁-C₂-alkyl)aminocarbonyl, C₁-C₂-alkoxy, CF₃, CHF₂, OCF₃ and OCHF₂.

In a further embodiment, two radicals R^{c} that are bound to adjacent ring member atoms of the Het group do not form together with said ring member atoms any fused cycle.

In one embodiment, R^{c} is halogen and preferably selected from fluorine and chlorine, and in particular, chlorine.

In a further embodiment, R^{c} is C₁-C₄-alkyl and preferably selected from methyl, ethyl, n-propyl and i-propyl, and in particular, methyl.

In a further embodiment, R^{c} is C₁-C₄-haloalkyl, more preferably, R^{c} is C₁-haloalkyl and in particular, trifluoromethyl.

In a further embodiment, R^{c} is C₁-C₄-alkoxy and preferably selected from methoxy and ethoxy.

In a further embodiment, R^{c} is C₁-C₄-haloalkoxy and preferably halomethoxy such as difluoromethoxy, trifluoromethoxy, dichloromethoxy and trichloromethoxy; haloethoxy such as 2,2-difluoroethoxy, 2,2,2-trifluoroethoxy, 2,2-dichloroethoxy and 2,2,2-trichloroethoxy; halo-n-propoxy, halo-i-propoxy, halo-n-butoxy, halo-1-methyl-propoxy, halo-2-methyl-propoxy or halo-1,1-dimethylethoxy.

In a further embodiment, R^{c} is C₃-C₈-cycloalkyl and preferably selected from cyclopropyl, cylopentyl and cyclohexyl, and in particular, cyclopropyl.

In a further embodiment, R^{c} is phenyl. In a further embodiment, R^{c} is phenoxy.

In a further embodiment, R^{c} is a 6-membered heteroaryl, wherein the ring member atoms of the heteroaryl include besides carbon atoms 1, 2, 3 or 4 heteroatoms selected from the group of N, O and S and is unsubstituted or carries 1, 2, 3 or 4 identical or different groups R^{d}.

Another embodiment relates to compounds I, wherein R^{c} is a 5-membered heteroaryl, wherein the ring member atoms of the heteroaryl include besides carbon atoms 1, 2, 3 or 4 heteroatoms selected from the group of N, O and S, and wherein R^{c} is unsubstituted or carries 1, 2, 3 or 4 identical or different groups R^{d}.

If R^{c} is C(═O)R′, R′ is selected from NH₂, C₁-C₄-alkyl, C₁-C₄-haloalkyl, C₁-C₄-alkoxy, C₁-C₄-alkoxy-C₁-C₄-alkoxy, C₁-C₄-haloalkoxy, C₁-C₄-alkylamino and di(C₁-C₄-alkyl)-amino.

If R^{c} is C(═NOR″)R′″, in one embodiment, R″ is C₁-C₄-alkyl, C₁-C₄-haloalkyl, C₂-C₄-alkenyl, C₂-C₄-alkynyl or C₁-C₄-alkoxy-C₁-C₄-alkyl.

If R^{c} is C(═NOR″)R′″, R″ is preferably C₁-C₄-alkyl and more preferably selected from methyl, ethyl, n-propyl, i-propyl, and in particular, R″ is methyl.

If R^{c} is C(═NOR″)R′″, R′″ is C₁-C₄-alkyl and preferably selected from methyl, ethyl, n-propyl, i-propyl, and in particular, R′″ is methyl. If R^{c} is C(═NOR″)R′″, in another embodiment, R′″ is hydrogen.

If R^{c} is present, one embodiment relates to compounds I, wherein R^{c} carries 1, 2, 3 or 4 radicals R^{d}, preferably 1, 2 or 3 radicals R^{d}, and more preferably 1 or 2 radicals R^{d}. In another embodiment, R^{c} carries one radical R^{d}. In a further embodiment, R^{c} carries two radicals R^{d}. In a further embodiment the group R^{c} carries three radicals R^{d}.

In one embodiment, R^{d} is halogen and preferably selected from fluorine and chlorine, and in particular, chlorine. In another embodiment, R^{d} is CN.

In a further embodiment, R^{d} is C₁-C₄-alkyl and preferably selected from methyl, ethyl, n-propyl and i-propyl and in particular, R^{d} is methyl.

In a further embodiment, R^{d} is C₁-C₄-haloalkyl. More preferably, R^{c} is C₁-haloalkyl and selected from fluoromethyl, difluoromethyl, trifluoromethyl, chloromethyl, dichloromethyl and trichloromethyl, and in particular, R^{d} is trifluoromethyl.

The compounds I can be prepared by various routes in analogy to prior art processes known per se for preparing sulfonamide compounds and, advantageously, by the synthesis shown in the following schemes and in the experimental part of this application.

Accordingly, a further aspect of the present invention relates to a process for preparing compounds of formula I as defined before, which comprises reacting aminomethylpyridine compounds of formula II, under basic conditions with sulfonic acid compounds of formula III, wherein the leaving group L is hydroxyl or halogen:

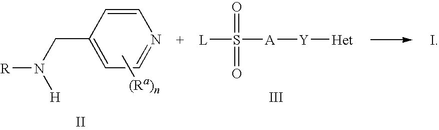

This reaction can be performed in accordance with standard methods of organic chemistry, see for example, Lieb. Ann. Chem. P. 641, 1990, or WO 05/033081.

This reaction is usually carried out in an inert organic solvent. Suitable solvents are aliphatic hydrocarbons, such as pentane, hexane, cyclohexane and petroleum ether, aromatic hydrocarbons, such as toluene, o-, m- and p-xylene, halogenated hydrocarbons, such as dichloromethane (DCM), chloroform and chlorobenzene, ethers, such as diethyl ether, diisopropyl ether, methyl tert.-butyl ether (MTBE), dioxane, anisole and tetrahydrofuran (THF), nitriles, such as acetonitrile and propionitrile, ketones, such as acetone, methyl ethyl ketone, diethyl ketone and tert.-butyl methyl ketone, and also dimethyl sulfoxide (DMSO), dimethylformamide (DMF) and dimethylacetamide, preferably THF, MTBE, DCM, chloroform, acetonitrile, toluene or DMF. It is also possible to use mixtures of the solvents mentioned.

The reaction is carried out in the presence of a base. Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal and alkaline earth metal carbonates, such as lithium carbonate, potassium carbonate and calcium carbonate, and also alkali metal bicarbonates, such as sodium bicarbonate, moreover organic bases, for example tertiary amines, such as trimethylamine, triethylamine, diisopropylethylamine and N-methylpiperidine, pyridine, substituted pyridines, such as collidine, lutidine and 4 dimethylaminopyridine, and also bicyclic amines. Particular preference is given to triethylamine, pyridine, triethylamine and potassium carbonate. The bases are generally employed in equimolar amounts, in excess or, if appropriate, as solvent. The amount of base is typically 0.5 to 5 molar equivalents relative to 1 mole of compounds II.

Generally, the reaction is carried out at temperatures of from −30° C. to 120° C., preferably from −10° C. to 100° C.

The starting materials, i.e. compounds II and compounds III, are generally reacted with one another in equimolar amounts.

Compounds II are known from the literature (e.g. from WO 06/097489, WO 02/066470, U.S. Pat. No. 4,482,437 or JP 04243867) or are commercially available or they can be prepared for example by reduction of the corresponding oximes IV.a, nitriles IV.b, or amides IV.c as described below. Appropriate methods therefor are known to those skilled in the art as shown below:

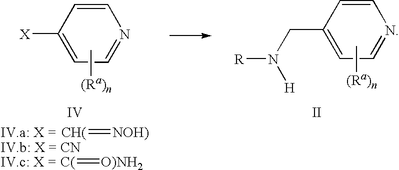

Methods suitable for the reduction of oxime compounds IV.a to the corresponding compounds II have been described in the literature e.g. in March, J. “Advanced Organic Chemistry: Reactions, Mechanisms, and Structure” (John Wiley & Sons, New York, 4th edition, 1992, pp. 1218-1219); J. Prak. Chem. Chem. Ztg. 695 (1974); or J. Org. Chem. 26, 1291 (1961).

Methods suitable for the reduction of nitrile compounds IV.b to the corresponding amine compounds II have been described in the literature, e.g. in March, J. “Advanced Organic Chemistry: Reactions, Mechanisms, and Structure” (John Wiley & Sons, New York, 4th edition, 1992, pp. 918-919); Heterocycl. 675 (1995); Heterocycl. 48 (6) 1203 (1998); or Chem. Pharm. Bull. 21, 1927 (1973).

Methods suitable for the reduction of amide compounds IV.c to the corresponding amine compounds II have been described in the literature, e.g. in March, J. “Advanced Organic Chemistry: Reactions, Mechanisms, and Structure” (John Wiley & Sons, New York, 4th edition, 1992, pp. 1212-1213 or U.S. Pat. No. 4,859,672.

Oxime compounds IV.a can be prepared for example from either the respective aldehyde compounds (X═CHO; compounds IV.d) or the methylderivatives (X═CH₃; compounds IV.e), in analogy to Houben-Weyl, vol. 10/4, Thieme, Stuttgart, 1968; vol. 11/2, 1957; vol E5, 1985; J. Prakt. Chem-Chem. Ztg. 336(8), 695-697, 1994; Tetrahedron Lett. 42(39), 6815-6818, 2001; or Heterocycl. 29(9), 1741-1760, 1989.

Aldehyde compounds IV.d can be synthesized from methyl compounds IV.e in analogy to J. Org. Chem. 51(4), 536-537, 1986, or from haloderivatives (X=halogen, compounds IV.f) as shown in Eur. J. Org. Chem. 8, pp. 1576-1588, 2003; Tetrahedron Lett. 40(19), 3719-3722, 1999; Tetrahedron Lett. 55(41), 12149-12156, 1999.

Nitrile compounds IV.b are either commercially available or can be prepared in analogie to methods described in J. Organometal. Chem. 689(24), 4576, 2004; Synth. Commun. 2391, 2002; Heterocycl. 41(4), 675, 1995; Chem. Pharm. Bull. 21, 1927 1973; or J. Chem. Soc. 426, 1942, e.g. from the corresponding halo compounds IV.f by reaction with CuCN, Zn(CN)₂ or potassium hexacyanoferrate (II). Compounds IV.f are either commercially available or can be synthesized according to standard methods. A further method for preparing nitrile compounds IV.b from the corresponding amide compounds IV.c is described in Synthesis 373 (2001) and Indian J. Chem. B. 40(8), 722 (2001).

Amide compounds IV.c can be prepared, for example, from the corresponding carboxylic acid chlorides by reaction with ammonia.

A further method to build up compounds II is shown below, wherein Boc is tert-butyloxycarbonyl:

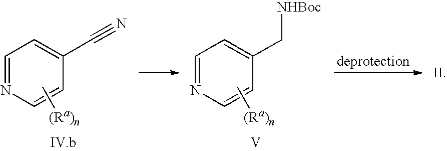

The hydrogenation of nitrile compounds IV.b in the presence of a catalyst, such as Raney nickel or palladium-on-carbon and t-butyl dicarbonate gives the N-protected compounds V. On treating with hydrogen bromide/glacial acetic acid or with trifluoroacetic acid containing water, the compounds V can be deprotected to yield compounds II.

Compounds II, wherein R^{a} is alkoxy, haloalkoxy, alkylthio or haloalkylthio, can be prepared in analogy to standard processes from compounds V, wherein R^{a} is halogen, especially chlorine, for example in analogy to methods described in J. Heterocycl. Chem. 42(7), 1369-1379, (2005); Tetrahedron Lett. 47(26), 4415-4418, 2006; or Chem. Pharm. Bull. 31(12), 4533-8, 1983. This synthesis route is shown below:

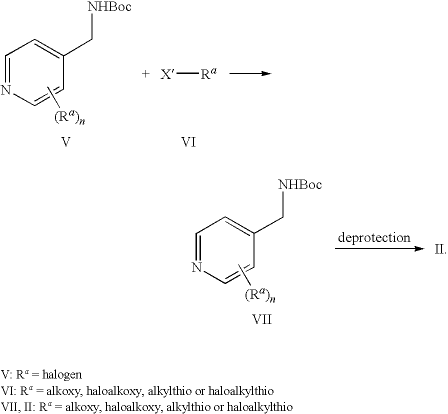

Compounds V are reacted with compounds X′—R^{a} (referred to as compounds VI) to give compounds VII. Depending on the R^{a} group to be introduced, the compounds VI are inorganic alkoxides, haloalkoxides, thiolates or halothiolates. The reaction is effected advantageously in an inert solvent. The cation X′ in formula VI is of little importance; for practical reasons, ammonium salts, tetraalkylammonium salts such as tetramethylammonium or tetraethylammonium salts, or alkali metal salts or alkaline earth metal salts are typically preferred. Suitable solvents comprise ethers such as dioxane, diethyl ether, MTBE and preferably THF, halogenated hydrocarbons such as DCM or dichloroethane, aromatic hydrocarbons such as toluene, and mixtures thereof. Deprotection of the amino group in formula VII to give the desired compounds II can be accomplished as described above for the deprotection of compounds V.

Compounds II, wherein R^{a} is alkyl, haloalkyl, alkenyl, alkynyl, cycloalkyl or alkyl-cycloalkyl, can advantageously be prepared by reacting compounds II, wherein R^{a} is halogen, with organometallic compounds R^{a}—Mt, wherein R^{a} is alkyl, haloalkyl, alkenyl, alkynyl, cycloalkyl or alkyl-cycloalkyl and Mt is lithium, magnesium or zinc. The reaction is effected preferably in the presence of catalytic or, in particular, at least equimolar amounts of transition metal salts and/or compounds, in particular in the presence of Cu salts such as Cu(I) halides and especially Cu(I) iodide, or Pd-catalyzed. The reaction is effected generally in an inert organic solvent, for example one of the aforementioned ethers, in particular THF, an aliphatic or cycloaliphatic hydrocarbon such as hexane, cyclohexane and the like, an aromatic hydrocarbon such as toluene, or in a mixture of these solvents. The temperatures required for this purpose are in the range of from −100 to +100° C. and especially in the range from −80° C. to +40° C.

Sulfonic acid compounds III are known from prior art or can be obtained according to procedures known in the art.

Two suitable methods to build up compounds III, wherein L is chlorine, are shown below in analogy to methods described in Chem. Soc., Chem. Commun. 118 (1970); U.S. Pat. No. 4,844,728; Russ. J. Org. Chem. 42(6) (2006); Tetrahedron Lett. 3559 (1997); J. Org. Chem. 1080 (2006); Heterocycles 36(6) 1317 (1994) U.S. Pat. No. 4,789,393; EP-A 995 750; WO 98/40332 and U.S. Pat. No. 4,668,279:

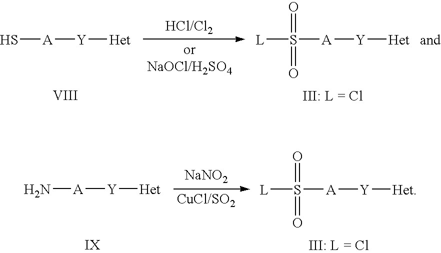

The starting materials VIII and IX are either commerically available or can be prepared in analogy to methods described in J. Heterocycl. Chem. 16, 169, 1979; Heterocycles 2131, 1982; or Heterocycles 117, 1984.

Compounds I, compounds II and compounds V, wherein R is hydrogen can be converted by conventional processes such as alkylation. Examples of suitable alkylating agents include alkyl halides, such as alkyl chloride, alkyl bromide or alkyl iodide, examples being methyl chloride, methyl bromide or methyl iodide, or dialkyl sulfates such as dimethyl sulfate or diethyl sulfate. The reaction with the alkylating agent is carried out advantageously in the presence of a solvent. Solvents used for these reactions are—depending on temperature range—aliphatic, cycloaliphatic or aromatic hydrocarbons such as hexane, cyclohexane, toluene, xylene, chlorinated aliphatic and aromatic hydrocarbons such as DCM, chlorobenzene, open-chain dialkyl ethers such as diethyl ether, di-n-propyl ether, MTBE, cyclic ethers such as THF, 1,4-dioxane, glycol ethers such as dimethyl glycol ether, or mixtures of these solvents.

The N-oxides may be prepared from compounds I according to conventional oxidation methods, for example by treating compounds I with an organic peracid such as metachloroperbenzoic acid (cf. J. Med. Chem. 38(11), 1892-903 (1995) or WO 03/64572); or with inorganic oxidizing agents such as hydrogen peroxide (cf. J. Heterocycl. Chem. 18(7), 1305-8 (1981)) or oxone (cf. J. Am. Chem. Soc. 123(25), 5962-5973 (2001)). The oxidation may lead to pure mono-N-oxides or to a mixture of different N-oxides, which can be separated by conventional methods such as chromatography.

If individual compounds I cannot be obtained by the routes described above, they can be prepared by derivatization of other compounds I.

If the synthesis yields mixtures of isomers, a separation is generally not necessarily required since in some cases the individual isomers can be interconverted during workup for use or during application (for example under the action of light, acids or bases). Such conversions may also take place after use, for example in the treatment of plants in the treated plant, or in the harmful fungus to be controlled.

The compounds I and the compositions according to the invention, respectively, are suitable as fungicides. They are distinguished by an outstanding effectiveness against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes and Deuteromycetes (syn. Fungi imperfecti). Some are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e.g. wheat, rye, barley, triticale, oats or rice; beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; natural rubber plants or ornamental and forestry plants, such as flowers, shrubs, broad-leaved trees or evergreens, e.g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transtional modification of protein(s), oligo- or polypeptides e.g. by glycosylation or polymer additions such as prenylated, acetylated or farnesylated moieties or PEG moieties.

Plants that have been modified by breeding, mutagenesis or genetic engineering, e.g. have been rendered tolerant to applications of specific classes of herbicides, such as hydroxyphenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/026390, WO 97/41218, WO 98/002526, WO 98/02527, WO 04/106529, WO 05/20673, WO 03/014357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A 242 236, EP-A 242 246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), e.g. Clearfield® summer rape (Canola, BASF SE, Germany) being tolerant to imidazolinones, e.g. imazamox. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, e.g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e.g. WO 02/015701). Further examples of such toxins or genetically modified plants capable of synthesizing such toxins are disclosed, e.g., in EP-A 374 753, WO 93/007278, WO 95/34656, EP-A 427 529, EP-A 451 878, WO 03/18810 and WO 03/52073. The methods for producing such genetically modified plants are generally known to the person skilled in the art and are described, e.g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e.g., described in the publications mentioned above, and some of which are commercially available such as YieldGard° (corn cultivars producing the Cry1Ab toxin), YieldGard° Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT° (cotton cultivars producing a VIP-toxin); NewLeaf® (potato cultivars producing the Cry3A toxin); Bt-Xtra®, NatureGard®, KnockOut°, BiteGard®, Protecta®, Bt11 (e.g. Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g. EP-A 392 225), plant disease resistance genes (e.g. potato cultivars, which express resistance genes acting against Phytophthora infestans derived from the mexican wild potato Solanum bulbocastanum) or T4-lysozym (e.g. potato cultivars capable of synthesizing these proteins with increased resistance against bacteria such as Erwinia amylvora). The methods for producing such genetically modified plants are generally known to the person skilled in the art and are described, e.g. in the publications mentioned above.

Furthermore, plants are also covered that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e.g. oil crops that produce health-promoting long-chain omega-3 fatty acids or unsaturated omega-9 fatty acids (e.g. Nexera® rape, DOW Agro Sciences, Canada).

Furthermore, plants are also covered that contain by the use of recombinant DNA techniques a modified amount of substances of content or new substances of content, specifically to improve raw material production, e.g. potatoes that produce increased amounts of amylopectin (e.g. Amflora® potato, BASF SE, Germany).

The compounds I and compositions thereof, respectively, are particularly suitable for controlling the following plant diseases:

Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Erysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporioides); Corticium spp., e.g. C. sasakii (sheath blight) on rice; Corynespora cassiicola (leaf spots) on soybeans and or namentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chiamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. graminearum or F. culmorum (root rot, scab or head blight) on cereals (e.g. wheat or barley), F. oxysporum on tomatoes, F. solani on soybeans and F. verticillioides on corn; Gaeumannomyces graminis (take-all) on cereals (e.g. wheat or barley) and corn; Gibberella spp. on cereals (e.g. G. zeae) and rice (e.g. G. fujikuroi: Bakanae disease); Glomerella cingulata on vines, pome fruits and other plants and G. gossypii on cotton; Grain-staining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or ‘rotbrenner’, anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondite (brown or leaf rust) on cereals, such as e.g. wheat, barley or rye, and asparagus (e.g. P. asparagi); Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnata (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Venturia spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

The compounds I and compositions thereof, resepctively, are also suitable for controlling harmful fungi in the protection of materials (e.g. wood, paper, paint dispersions, fiber or fabrics) and in the protection of stored products. As to the protection of wood and construction materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products the following yeast fungi are worthy of note: Candida spp. and Saccharomyces cerevisae.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e.g. increased biomass and/or increased content of valuable ingredients), plant vigor (e.g. improved plant growth and/or greener leaves (“greening effect”)), quality (e.g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising a solvent or solid carrier and at least one compound I and to the use for controlling harmful fungi.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e.g. solutions, emulsions, suspensions, dusts, powders, pastes and granules. The composition type depends on the particular intended purpose; in each case, it should ensure a fine and uniform distribution of the compound according to the invention.

Examples for composition types are suspensions (SC, OD, FS), emulsifiable concentrates (EC), emulsions (EW, EO, ES), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF).

Usually the composition types (e.g. SC, OD, FS, EC, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalene-sulfonic acid (Nekal® types, BASF, Germany), and fatty acids, alkylsulfonates, alkyl-arylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof.

Examples for thickeners (i.e. compounds that impart a modified flowability to compositions, i.e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Bactericides may be added for preservation and stabilization of the composition. Examples for suitable bactericides are those based on dichlorophene and benzyl-alcohol hemi formal (Proxel® from ICI or Acticide® RS from Thor Chemie and Kathon®MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as e.g. Silikon®SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned and the designations rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

### Examples for Composition Types are:

1. Composition Types for Dilution with Water

i) Water-Soluble Concentrates (SL, LS)

10 parts by weight of a compound I according to the invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

ii) Dispersible Concentrates (DC)

20 parts by weight of a compound I according to the invention are dissolved in 70 parts by weight of cyclohexanone with addition of 10 parts by weight of a dispersant, e.g. polyvinylpyrrolidone. Dilution with water gives a dispersion. The active substance content is 20% by weight.

iii) Emulsifiable Concentrates (EC)

15 parts by weight of a compound I according to the invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

iv) Emulsions (EW, EO, ES)

25 parts by weight of a compound I according to the invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

v) Suspensions (SC, OD, FS)

In an agitated ball mill, 20 parts by weight of a compound I according to the invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

vi) Water-Dispersible Granules and Water-Soluble Granules (WG, SG)

50 parts by weight of a compound I according to the invention are ground finely with addition of 50 parts by weight of dispersants and wetting agents and prepared as water-dispersible or water-soluble granules by means of technical appliances (e.g. extrusion, spray tower, fluidized bed). Dilution with water gives a stable dispersion or solution of the active substance. The composition has an active substance content of 50% by weight.

vii) Water-Dispersible Powders and Water-Soluble Powders (WP, SP, SS, WS)

75 parts by weight of a compound I according to the invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a compound I according to the invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

### 2. Composition Types to be Applied Undiluted

ix) Dustable Powders (DP, DS)

5 parts by weight of a compound I according to the invention are ground finely and mixed intimately with 95 parts by weight of finely divided kaolin. This gives a dustable composition having an active substance content of 5% by weight.

x) Granules (GR, FG, GG, MG)

0.5 parts by weight of a compound I according to the invention is ground finely and associated with 99.5 parts by weight of carriers. Current methods are extrusion, spray-drying or the fluidized bed. This gives granules to be applied undiluted having an active substance content of 0.5% by weight.

xi) ULV Solutions (UL)

10 parts by weight of a compound I according to the invention are dissolved in 90 parts by weight of an organic solvent, e.g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum).

Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typically, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water.

The active substances can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

When used in the protection of materials or stored products, the amount of active substance applied depends on the kind of application area and on the desired effect. Amounts customarily applied in the protection of materials are, e.g., 0.001 g to 2 kg, preferably 0.005 g to 1 kg, of active substance per cubic meter of treated material.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

The compositions according to the invention can, in the use form as fungicides, also be present together with other active substances, e.g. with herbicides, insecticides, growth regulators, fungicides or else with fertilizers, as pre-mix or, if appropriate, not until immediately prior to use (tank mix).

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The following list of active substances, in conjunction with which the compounds according to the invention can be used, is intended to illustrate the possible combinations but does not limit them:

### A) Strobilurins

azoxystrobin, dimoxystrobin, enestroburin, fluoxastrobin, kresoxim-methyl, metominostrobin, orysastrobin, picoxystrobin, pyraclostrobin, pyribencarb, trifloxystrobin, 2-(2-(6-(3-chloro-2-methyl-phenoxy)-5-fluoro-pyrimidin-4-yloxy)-phenyl)-2-methoxy-imino-N-methyl-acetamide, 3-methoxy-2-(2-(N-(4-methoxy-phenyl)-cyclopropane-carboximidoylsulfanylmethyl)-phenyl)-acrylic acid methyl ester, methyl (2-chloro-5-[1-(3-methylbenzyloxyimino)ethyl]benzyl)carbamate and 2-(2-(3-(2,6-di-chlorophenyl)-1-methyl-allylideneaminooxymethyl)-phenyl)-2-methoxyimino-N-methyl-acetamide;

### B) Carboxamides

carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methyl-thiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide, N-(4′-trifluoro-methylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H-pyrazole-4-carboxamide and N-(2-(1,3,3-trimethyl-butyl)-phenyl)-1,3-dimethyl-5-fluoro-1H-pyrazole-4-carboxamide;
carboxylic morpholides: dimethomorph, flumorph, pyrimorph;
benzoic acid amides: flumetover, fluopicolide, fluopyram, zoxamide, N-(3-Ethyl-3,5,5-trimethyl-cyclohexyl)-3-formylamino-2-hydroxy-benzamide;
other carboxamides: carpropamid, dicyclomet, mandiproamid, oxytetracyclin, silthio-farm and N-(6-methoxy-pyridin-3-yl)cyclopropanecarboxylic acid amide;

### C) Azoles

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, 1-(4-chloro-phenyl)-2-([1,2,4]triazol-1-yl)-cycloheptanol;
imidazoles: cyazofamid, imazalil, pefurazoate, prochloraz, triflumizol;
benzimidazoles: benomyl, carbendazim, fuberidazole, thiabendazole;
others: ethaboxam, etridiazole, hymexazole and 2-(4-chloro-phenyl)-N-[4-(3,4-di-methoxy-phenyl)-isoxazol-5-yl]-2-prop-2-ynyloxy-acetamide;

### D) Heterocyclic Compounds

pyridines: fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbo-nitrile, N-(1-(5-bromo-3-chloro-pyridin-2-yl)-ethyl)-2,4-dichloronicotinamide, N-[(5-bromo-3-chloro-pyridin-2-yl)-methyl]-2,4-dichloro-nicotinamide;
pyrimidines: bupirimate, cyprodinil, diflumetorim, fenarimol, ferimzone, mepanipyrim, nitrapyrin, nuarimol, pyrimethanil;
piperazines: triforine;
pyrroles: fenpiclonil, fludioxonil;
morpholines: aldimorph, dodemorph, dodemorph-acetate, fenpropimorph, tridemorph;
piperidines: fenpropidin;
dicarboximides: fluoroimid, iprodione, procymidone, vinclozolin;
non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester;
others: acibenzolar-5-methyl, amisulbrom, anilazin, blasticidin-S, captafol, captan, chinomethionat, dazomet, debacarb, diclomezine, difenzoquat, difenzoquat-methyl-sulfate, fenoxanil, Folpet, oxolinic acid, piperalin, proquinazid, pyroquilon, quinoxyfen, triazoxide, tricyclazole, 2-butoxy-6-iodo-3-propylchromen-4-one, 5-chloro-5-chloro-7-(4-methyl-piperidin-1-yl)-6-(2,4,6-trifluorophenyl)-[1,2,4]-titriazolo[1,5-a]pyrimidine and 5-ethyl-6-octyl-[1,2,4]-triazolo[1,5-a]pyrimidine-7-ylamine;

### E) Carbamates

thio- and dithiocarbamates: ferbam, mancozeb, maneb, metam, methasulphocarb, metiram, propineb, thiram, zineb, ziram;
carbamates: benthiavalicarb, diethofencarb, iprovalicarb, propamocarb, propamocarb hydrochlorid, valiphenal and N-(1-(1-(4-cyano-phenyl)ethanesulfonyl)-but-2-yl) carbamic acid-(4-fluorophenyl) ester;

### F) Other Active Substances

guanidines: guanidine, dodine, dodine free base, guazatine, guazatine-acetate, iminoctadine, iminoctadine-triacetate, iminoctadine-tris(albesilate);
antibiotics: kasugamycin, kasugamycin hydrochloride-hydrate, streptomycin, polyoxine, validamycin A;
nitrophenyl derivates: binapacryl, dinobuton, dinocap, nitrthal-isopropyl, tecnazen, organometal compounds: fentin salts, such as fentin-acetate, fentin chloride or fentin hydroxide;
sulfur-containing heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide;
inorganic active substances: Bordeaux mixture, copper acetate, copper hydroxide, copper oxychloride, basic copper sulfate, sulfur;
others: biphenyl, bronopol, cyflufenamid, cymoxanil, diphenylamin, metrafenone, mildiomycin, oxin-copper, prohexadione-calcium, spiroxamine, tolylfluanid, N-(cyclo-propylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethyl-silanyl-propoxy)-phenyl)-N-ethyl-N-methyl formamidine, N′-(5-difluoromethyl-2-methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, acetic acid 6-tert.-butyl-8-fluoro-2,3-dimethyl-quinolin-4-yl ester and methoxy-acetic acid 6-tert-butyl-8-fluoro-2,3-dimethyl-quinolin-4-yl ester.

### G) Growth Regulators

abscisic acid, amidochlor, ancymidol, 6-benzylaminopurine, brassinolide, butralin, chiormequat (chiormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N-6-benzyladenine, paclobutrazol, prohexadione (prohexadione-calcium), prohydrojasmon, thidiazuron, triapenthenol, tributyl phosphorotrithioate, 2,3,5-tri-iodobenzoic acid, trinexapac-ethyl and uniconazole;

### H) Herbicides

acetamides: acetochlor, alachlor, butachlor, dimethachlor, dimethenamid, flufenacet, mefenacet, metolachior, metazachlor, napropamide, naproanilide, pethoxamid, pretilachlor, propachior, thenylchlor;
amino acid derivatives: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
Bipyridyls: diquat, paraquat;
(thio)carbamates: asulam, butylate, carbetamide, desmedipham, dimepiperate, eptam (EPIC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethaifluralin, oryzalin, pendimethalin, prodiamine, trifluralin;
diphenyl ethers: acifluorfen, aclonifen, bifenox, diclofop, ethoxyfen, fomesafen, lactofen, oxyfluorfen;
hydroxybenzonitriles: bomoxynil, dichlobenil, ioxynil;
imidazolinones: imazamethabenz, imazamox, imazapic, imazapyr, imazaquin, imazethapyr;
phenoxy acetic acids: clomeprop, 2,4-dichlorophenoxyacetic acid (2,4-D), 2,4-DB, dichlorprop, MCPA, MCPA-thioethyl, MCPB, Mecoprop;
pyrazines: chloridazon, flufenpyr-ethyl, fluthiacet, norflurazon, pyridate;
pyridines: aminopyralid, clopyralid, diflufenican, dithiopyr, fluridone, fluoroxypyr, picloram, picolinafen, thiazopyr;
sulfonyl ureas: amidosulfuron, azimsulfuron, bensulfuron, chlorimuron-ethyl, chlorsulfuron, cinosulfuron, cyclosulfamuron, ethoxysulfuron, flazasulfuron, flucetosulfuron, flupyrsulfuron, foramsulfuron, halosulfuron, imazosulfuron, iodosulfuron, mesosulfuron, metsulfuron-methyl, nicosulfuron, oxasulfuron, primisulfuron, prosulfuron, pyrazosulfuron, rimsulfuron, sulfometuron, sulfosulfuron, thifensulfuron, triasulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxy-pyrimidin-2-yl)urea;
triazines: ametryn, atrazine, cyanazine, dimethametryn, ethiozin, hexazinone, metamitron, metribuzin, prometryn, simazine, terbuthylazine, terbutryn, triaziflam;
ureas: chlorotoluron, daimuron, diuron, fluometuron, isoproturon, linuron, metha-benzthiazuron, tebuthiuron;
other acetolactate synthase inhibitors: bispyribac-sodium, cloransulam-methyl, di-closulam, florasulam, flucarbazone, flumetsulam, metosulam, ortho-sulfamuron, penoxsulam, propoxycarbazone, pyribambenz-propyl, pyribenzoxim, pyriftalid, pyrimi-nobac-methyl, pyrimisulfan, pyrithiobac, pyroxasulfone, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamide, dicamba, difenzoquat, diflufenzopyr, Drechslera monoceras, endothal, ethofumesate, etobenzanid, fentrazamide, flumiclorac-pentyl, flumioxazin, flupoxam, fluorochloridone, flurtamone, indanofan, isoxaben, isoxaflutole, lenacil, propanil, propyzamide, quinclorac, quinmerac, mesotrione, methyl arsonic acid, naptalam, oxadiargyl, oxadiazon, oxaziclomefone, pentoxazone, pinoxaden, pyraclonil, pyraflufen-ethyl, pyrasulfotole, pyrazoxyfen, pyrazolynate, quinoclamine, saflufenacil, sulcotrione, sulfentrazone, terbacil, tefuryltrione, tembotrione, thiencarbazone, topramezone, 4-hydroxy-3-[2-(2-methoxy-ethoxymethyl)-6-trifluoromethyl-pyridine-3-carbonyl]-bicyclo[3.2.1]oct-3-en-2-one, (3-[2-chloro-4-fluoro-5-(3-methyl-2,6-dioxo-4-trifluoromethyl-3,6-dihydro-2H-pyrimidin-1-yl)-phenoxy]-pyridin-2-yloxy)-acetic acid ethyl ester, 6-amino-5-chloro-2-cyclopropyl-pyrimidine-4-carboxylic acid methyl ester, 6-chloro-3-(2-cyclopropyl-6-methyl-phenoxy)-pyridazin-4-ol, 4-amino-3-chloro-6-(4-chloro-phenyl)-5-fluoro-pyridine-2-carboxylic acid, 4-amino-3-chloro-6-(4-chloro-2-fluoro-3-methoxy-phenyl)-pyridine-2-carboxylic acid methyl ester, and 4-amino-3-chloro-6-(4-chloro-3-dimethylamino-2-fluoro-phenyl)-pyridine-2-carboxylic acid methyl ester.

### I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoate, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, sulprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfen-valerate, etofenprox, fenpropathrin, fenvalerate, imiprothrin, lambda-cyhalothrin, permethrin, prallethrin, pyrethrin I and II, resmethrin, silafluofen, tau-fluvalinate, tefluthrin, tetramethrin, tralomethrin, transfluthrin, profluthrin, dimefluthrin;
insect growth regulators: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramat;
nicotinic receptor agonists/antagonists compounds: clothianidin, dinotefuran, imidacloprid, thiamethoxam, nitenpyram, acetamiprid, thiacloprid, 1-(2-chloro-thiazol-5-ylmethyl)-2-nitrimino-3,5-dimethyl-[1,3,5]triazinane;
GABA antagonist compounds: endosulfan, ethiprole, fipronil, vaniliprole, pyrafluprole, pyriprole, 5-amino-1-(2,6-dichloro-4-methyl-phenyl)-4-sulfinamoyl-1H-pyrazole-3-carbothioic acid amide;
macrocyclic lactone insecticides: abamectin, emamectin, milbemectin, lepimectin, spinosad, spinetoram;
mitochondrial electron transport inhibitor (METI) I acaricides: fenazaquin, pyridaben, tebufenpyrad, tolfenpyrad, flufenerim;
METI II and III compounds: acequinocyl, fluacyprim, hydramethylnon;
Uncouplers: chlorfenapyr;
oxidative phosphorylation inhibitors: cyhexatin, diafenthiuron, fenbutatin oxide, propargite;
moulting disruptor compounds: cryomazine;
mixed function oxidase inhibitors: piperonyl butoxide;
sodium channel blockers: indoxacarb, metaflumizone;
others: benclothiaz, bifenazate, cartap, flonicamid, pyridalyl, pymetrozine, sulfur, thiocyclam, flubendiamide, chlorantraniliprole, cyazypyr (HGW86), cyenopyrafen, flupyrazofos, cyflumetofen, amidoflumet, imicyafos, bistrifluoron, and pyrifluquinazon.

The present invention furthermore relates to agrochemical compositions comprising a mixture of at least one compound I (component 1) and at least one further active substance useful for plant protection, e.g. selected from the groups A) to I) (component 2), in particular one further fungicide, e.g. one or more fungicide from the groups A) to F), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to F), as described above, is more efficient than combating those fungi with individual compounds I or individual fungicides from groups A) to F). By applying compounds I together with at least one active substance from groups A) to I) a synergistic effect can be obtained, i.e. more then simple addition of the individual effects is obtained (synergistic mixtures).

According to this invention, applying the compounds I together with at least one further active substance is to be understood to denote, that at least one compound of formula I and at least one further active substance occur simultaneously at the site of action (i.e. the harmful fungi to be controlled or their habitats such as infected plants, plant propagation materials, particularly seeds, surfaces, materials or the soil as well as plants, plant propagation materials, particularly seeds, soil, surfaces, materials or rooms to be protected from fungal attack) in a fungicidally effective amount. This can be obtained by applying the compounds I and at least one further active substance simultaneously, either jointly (e.g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

In binary mixtures, i.e. compositions according to the invention comprising one compound I (component 1) and one further active substance (component 2), e.g. one active substance from groups A) to I), the weight ratio of component 1 and component 2 generally depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:3 to 3:1.

In ternary mixtures, i.e. compositions according to the invention comprising one compound I (component 1) and a first further active substance (component 2) and a second further active substance (component 3), e.g. two active substances from groups A) to I), the weight ratio of component 1 and component 2 depends from the properties of the active substances used, preferably it is in the range of from 1:50 to 50:1 and particularly in the range of from 1:10 to 10:1, and the weight ratio of component 1 and component 3 preferably is in the range of from 1:50 to 50:1 and particularly in the range of from 1:10 to 10:1.

The components can be used individually or already partially or completely mixed with one another to prepare the composition according to the invention. It is also possible for them to be packaged and used further as combination composition such as a kit of parts.

In one embodiment of the invention, the kits may include one or more, including all, components that may be used to prepare a subject agrochemical composition. E. g., kits may include one or more fungicide component(s) and/or an adjuvant component and/or a insecticide component and/or a growth regulator component and/or a herbicide. One or more of the components may already be combined together or pre-formulated. In those embodiments where more than two components are provided in a kit, the components may already be combined together and as such are packaged in a single container such as a vial, bottle, can, pouch, bag or canister. In other embodiments, two or more components of a kit may be packaged separately, i.e., not pre-formulated. As such, kits may include one or more separate containers such as vials, cans, bottles, pouches, bags or canisters, each container containing a separate component for an agrochemical composition. In both forms, a component of the kit may be applied separately from or together with the further components or as a component of a combination composition according to the invention for preparing the composition according to the invention.

The user applies the composition according to the invention usually from a predosage device, a knapsack sprayer, a spray tank or a spray plane. Here, the agrochemical composition is made up with water and/or buffer to the desired application concentration, it being possible, if appropriate, to add further auxiliaries, and the ready-to-use spray liquor or the agrochemical composition according to the invention is thus obtained. Usually, 50 to 500 liters of the ready-to-use spray liquor are applied per hectare of agricultural useful area, preferably 100 to 400 liters.

According to one embodiment, individual components of the composition according to the invention such as parts of a kit or parts of a binary or ternary mixture may be mixed by the user himself in a spray tank and further auxiliaries may be added, if appropriate (tank mix).

In a further embodiment, either individual components of the composition according to the invention or partially premixed components, e.g. components comprising compounds I and/or active substances from the groups A) to I), may be mixed by the user in a spray tank and further auxiliaries and additives may be added, if appropriate (tank mix).

In a further embodiment, either individual components of the composition according to the invention or partially premixed components, e.g. components comprising compounds I and/or active substances from the groups A) to I), can be applied jointly (e.g. after tank mix) or consecutively.

Preference is also given to mixtures comprising a compound I (component 1) and at least one active substance selected from the strobilurines of group A) (component 2) and particularly selected from azoxystrobin, dimoxystrobin, fluoxastrobin, kresoxim-methyl, orysastrobin, picoxystrobin, pyraclostrobin and trifloxystrobin.

Preference is also given to mixtures comprising a compound I (component 1) and at least one active substance selected from the carboxamides of group B) (component 2) and particularly selected from bixafen, boscalid, sedaxane, fenhexamid, metalaxyl, isopyrazam, mefenoxam, ofurace, dimethomorph, flumorph, fluopicolid (picobenzamid), zoxamide, carpropamid, mandipropamid and N-(3′,4′,5′-trifluorobiphenyl-2-yl)-3-di-fluoromethyl-1-methyl-1H-pyrazole-4-carboxamide.

Preference is given to mixtures comprising a compound of formula I (component 1) and at least one active substance selected from the azoles of group C) (component 2) and particularly selected from cyproconazole, difenoconazole, epoxiconazole, fluquin-conazole, flusilazole, flutriafol, metconazole, myclobutanil, penconazole, propiconazole, prothioconazole, triadimefon, triadimenol, tebuconazole, tetraconazole, triticonazole, prochloraz, cyazofamid, benomyl, carbendazim and ethaboxam.

Preference is also given to mixtures comprising a compound I (component 1) and at least one active substance selected from the heterocyclic compounds of group D) (component 2) and particularly selected from fluazinam, cyprodinil, fenarimol, mepanipyrim, pyrimethanil, triforine, fludioxonil, dodemorph, fenpropimorph, tridemorph, fenpropidin, iprodione, vinclozolin, famoxadone, fenamidone, probenazole, proquinazid, acibenzolar-S-methyl, captafol, folpet, fenoxanil, quinoxyfen and 5-ethyl-6-octyl-[1,2,4]triazolo[1,5-a]pyrimidine-7-ylamine.

Preference is also given to mixtures comprising a compound I (component 1) and at least one active substance selected from the carbamates of group E) (component 2) and particularly selected from mancozeb, metiram, propineb, thiram, iprovalicarb, benthiavalicarb and propamocarb.

Preference is also given to mixtures comprising a compound I (component 1) and at least one active substance selected from the fungicides given in group F) (component 2) and particularly selected from dithianon, fentin salts, such as fentin acetate, fosetyl, fosetyl-aluminium, H₃PO₃ and salts thereof, chlorthalonil, dichlofluanid, thiophanat-methyl, copper acetate, copper hydroxide, copper oxychloride, copper sulfate, sulfur, cymoxanil, metrafenone and spiroxamine.

Accordingly, the present invention furthermore relates to compositions comprising one compound I (component 1) and one further active substance (component 2), which further active substance is selected from the column “Component 2” of the lines B-1 to B-346 of Table B.

A further embodiment relates to the compositions B-1 to B-346 listed in Table B, where a row of Table B corresponds in each case to a fungicidal composition comprising one of the in the present specification individualized compounds of formula I (component 1) and the respective further active substance from groups A) to I) (component 2) stated in the row in question. Preferably, the compositions described comprise the active substances in synergistically effective amounts.

TABLE B
Composition comprising one indiviualized compound I and one further active
substance from groups A) to I)

| Mixture | Component 1 | Component 2 |
|---|---|---|
| B-1 | one individualized compound I | Azoxystrobin |
| B-2 | one individualized compound I | Dimoxystrobin |
| B-3 | one individualized compound I | Enestroburin |
| B-4 | one individualized compound I | Fluoxastrobin |
| B-5 | one individualized compound I | Kresoxim-methyl |
| B-6 | one individualized compound I | Metominostrobin |
| B-7 | one individualized compound I | Orysastrobin |
| B-8 | one individualized compound I | Picoxystrobin |
| B-9 | one individualized compound I | Pyraclostrobin |
| B-10 | one individualized compound I | Pyribencarb |
| B-11 | one individualized compound I | Trifloxystrobin |
| B-12 | one individualized compound I | 2-(2-(6-(3-Chloro-2-methyl-phenoxy)- |
| | | 5-fluoro-pyrimidin-4-yloxy)-phenyl)-2- |
| | | methoxyimino-N-methyl-acetamide |
| B-13 | one individualized compound I | 2-(ortho-((2,5-Dimethylphenyl-oxy- |
| | | methylen)phenyl)-3-methoxy- |
| | | acrylsauremethylester |
| B-14 | one individualized compound I | 3-Methoxy-2-(2-(N-(4-methoxy-phenyl)- |
| | | cyclopropanecarboximidoylsulfanyl- |
| | | methyl)-phenyl)-acrylic acid methyl ester |
| B-15 | one individualized compound I | 2-(2-(3-(2,6-dichlorophenyl)-1-methyl- |
| | | allylideneaminooxymethyl)-phenyl)- |
| | | 2-methoxyimino-N-methyl-acetamide |
| B-16 | one individualized compound I | Benalaxyl |
| B-17 | one individualized compound I | Benalaxyl-M |
| B-18 | one individualized compound I | Benodanil |
| B-19 | one individualized compound I | Bixafen |
| B-20 | one individualized compound I | Boscalid |
| B-21 | one individualized compound I | Carboxin |
| B-22 | one individualized compound I | Fenfuram |
| B-23 | one individualized compound I | Fenhexamid |
| B-24 | one individualized compound I | Flutolanil |
| B-25 | one individualized compound I | Furametpyr |
| B-26 | one individualized compound I | Isopyrazam |
| B-27 | one individualized compound I | Isotianil |
| B-28 | one individualized compound I | Kiralaxyl |
| B-29 | one individualized compound I | Mepronil |
| B-30 | one individualized compound I | Metalaxyl |
| B-31 | one individualized compound I | Metalaxyl-M |
| B-32 | one individualized compound I | Ofurace |
| B-33 | one individualized compound I | Oxadixyl |
| B-34 | one individualized compound I | Oxycarboxin |
| B-35 | one individualized compound I | Penthiopyrad |
| B-36 | one individualized compound I | Sedaxane |
| B-37 | one individualized compound I | Tecloftalam |
| B-38 | one individualized compound I | Thifluzamide |
| B-39 | one individualized compound I | Tiadinil |
| B-40 | one individualized compound I | 2-Amino-4-methyl-thiazole-5-carboxylic |
| | | acid anilide |
| B-41 | one individualized compound I | 2-Chloro-N-(1,1,3-trimethyl-indan-4-yl)- |
| | | nicotinamide |
| B-42 | one individualized compound I | N-(3′,4′,5′-trifluorobiphenyl-2-yl)-3-di- |
| | | fluoromethyl-1-methy1-1H-pyrazole- |
| | | 4-carboxamide |
| B-43 | one individualized compound I | N-(4′-trifluoromethylthiobiphenyl-2-yl)- |
| | | 3-difluoromethyl-1-methyl-1H-pyrazole- |
| | | 4-carboxamide |
| B-44 | one individualized compound I | N-(2-(1,3-dimethyl-butyl)-phenyl)- |
| | | 1,3-dimethyl-5-fluoro-1H-pyrazole- |
| | | 4-carboxamide |
| B-45 | one individualized compound I | N-(2-(1,3,3-trimethyl-butyl)-phenyl)- |
| | | 1,3-dimethyl-5-fluoro-1H-pyrazole- |
| | | 4-carboxamide |
| B-46 | one individualized compound I | Dimethomorph |
| B-47 | one individualized compound I | Flumorph |
| B-48 | one individualized compound I | Pyrimorph |
| B-49 | one individualized compound I | Flumetover |
| B-50 | one individualized compound I | Fluopicolide |
| B-51 | one individualized compound I | Fluopyram |
| B-52 | one individualized compound I | Zoxamide |
| B-53 | one individualized compound I | N-(3-Ethyl-3,5,5-trimethyl-cyclohexyl)- |
| | | 3-formylamino-2-hydroxy-benzamide |
| B-54 | one individualized compound I | Carpropamid |
| B-55 | one individualized compound I | Diclocymet |
| B-56 | one individualized compound I | Mandipropamid |
| B-57 | one individualized compound I | Oxytetracyclin |
| B-58 | one individualized compound I | Silthiofam |
| B-59 | one individualized compound I | N-(6-methoxy-pyridin-3-yl)cyclopro- |
| | | panecarboxylic acid amide |
| B-60 | one individualized compound I | Azaconazole |
| B-61 | one individualized compound I | Bitertanol |
| B-62 | one individualized compound I | Bromuconazole |
| B-63 | one individualized compound I | Cyproconazole |
| B-64 | one individualized compound I | Difenoconazole |
| B-65 | one individualized compound I | Diniconazole |
| B-66 | one individualized compound I | Diniconazole-M |
| B-67 | one individualized compound I | Epoxiconazole |
| B-68 | one individualized compound I | Fenbuconazole |
| B-69 | one individualized compound I | Fluquinconazole |
| B-70 | one individualized compound I | Flusilazole |
| B-71 | one individualized compound I | Flutriafol |
| B-72 | one individualized compound I | Hexaconazol |
| B-73 | one individualized compound I | Imibenconazole |
| B-74 | one individualized compound I | Ipconazole |
| B-75 | one individualized compound I | Metconazole |
| B-76 | one individualized compound I | Myclobutanil |
| B-77 | one individualized compound I | Oxpoconazol |
| B-78 | one individualized compound I | Paclobutrazol |
| B-79 | one individualized compound I | Penconazole |
| B-80 | one individualized compound I | Propiconazole |
| B-81 | one individualized compound I | Prothioconazole |
| B-82 | one individualized compound I | Simeconazole |
| B-83 | one individualized compound I | Tebuconazole |
| B-84 | one individualized compound I | Tetraconazole |
| B-85 | one individualized compound I | Triadimefon |
| B-86 | one individualized compound I | Triadimenol |
| B-87 | one individualized compound I | Triticonazole |
| B-88 | one individualized compound I | Uniconazole |
| B-89 | one individualized compound I | 1-(4-Chloro-phenyl)-2-([1,2,4]triazol-1- |
| | | yl)-cycloheptanol |
| B-90 | one individualized compound I | Cyazofamid |
| B-91 | one individualized compound I | Imazalil |
| B-92 | one individualized compound I | Imazalil-sulfate |
| B-93 | one individualized compound I | Pefurazoate |
| B-94 | one individualized compound I | Prochloraz |
| B-95 | one individualized compound I | Triflumizole |
| B-96 | one individualized compound I | Benomyl |
| B-97 | one individualized compound I | Carbendazim |
| B-98 | one individualized compound I | Fuberidazole |
| B-99 | one individualized compound I | Thiabendazole |
| B-100 | one individualized compound I | Ethaboxam |
| B-101 | one individualized compound I | Etridiazole |
| B-102 | one individualized compound I | Hymexazole |
| B-103 | one individualized compound I | 2-(4-Chloro-phenyl)-N-[4-(3,4-dimethoxy- |
| | | phenyl)-isoxazol-5-yl]-2-prop-2-yn- |
| | | yloxy-acetamide |
| B-104 | one individualized compound I | Fluazinam |
| B-105 | one individualized compound I | Pyrifenox |
| B-106 | one individualized compound I | 3-[5-(4-Chloro-phenyl)-2,3-dimethyl- |
| | | isoxazolidin-3-yl]-pyridine |
| B-107 | one individualized compound I | 3-[5-(4-Methyl-phenyl)-2,3-dimethyl- |
| | | isoxazolidin-3-yl]-pyridine |
| B-108 | one individualized compound I | 2,3,5,6-Tetrachloro-4-methanesulfonyl- |
| | | pyridine |
| B-109 | one individualized compound I | 3,4,5-Trichloro-pyridine-2,6- |
| | | dicarbonitrile |
| B-110 | one individualized compound I | N-(1-(5-Bromo-3-chloro-pyridin-2-yl)- |
| | | ethyl)-2,4-dichloro-nicotinamide |
| B-111 | one individualized compound I | N-((5-Bromo-3-chloro-pyridin-2-yl)- |
| | | methyl)-2,4-dichloro-nicotinamide |
| B-112 | one individualized compound I | Bupirimate |
| B-113 | one individualized compound I | Cyprodinil |
| B-114 | one individualized compound I | Diflumetorim |
| B-115 | one individualized compound I | Fenarimol |
| B-116 | one individualized compound I | Ferimzone |
| B-117 | one individualized compound I | Mepanipyrim |
| B-118 | one individualized compound I | Nitrapyrin |
| B-119 | one individualized compound I | Nuarimol |
| B-120 | one individualized compound I | Pyrimethanil |
| B-121 | one individualized compound I | Triforine |
| B-122 | one individualized compound I | Fenpiclonil |
| B-123 | one individualized compound I | Fludioxonil |
| B-124 | one individualized compound I | Aldimorph |
| B-125 | one individualized compound I | Dodemorph |
| B-126 | one individualized compound I | Dodemorph-acetate |
| B-127 | one individualized compound I | Fenpropimorph |
| B-128 | one individualized compound I | Tridemorph |
| B-129 | one individualized compound I | Fenpropidin |
| B-130 | one individualized compound I | Fluoroimid |
| B-131 | one individualized compound I | Iprodione |
| B-132 | one individualized compound I | Procymidone |
| B-133 | one individualized compound I | Vinclozolin |
| B-134 | one individualized compound I | Famoxadone |
| B-135 | one individualized compound I | Fenamidone |
| B-136 | one individualized compound I | Flutianil |
| B-137 | one individualized compound I | Octhilinone |
| B-138 | one individualized compound I | Probenazole |
| B-139 | one individualized compound I | 5-Amino-2-iso-propyl-4-ortho-tolyl-2,3- |
| | | dihydro-pyrazole-1-carbothioic acid S- |
| | | allyl ester |
| B-140 | one individualized compound I | Acibenzolar-S-methyl |
| B-141 | one individualized compound I | Amisulbrom |
| B-142 | one individualized compound I | Anilazin |
| B-143 | one individualized compound I | Blasticidin-S |
| B-144 | one individualized compound I | Captafol |
| B-145 | one individualized compound I | Captan |
| B-146 | one individualized compound I | Chinomethionat |
| B-147 | one individualized compound I | Dazomet |
| B-148 | one individualized compound I | Debacarb |
| B-149 | one individualized compound I | Diclomezine |
| B-150 | one individualized compound I | Difenzoquat, |
| B-151 | one individualized compound I | Difenzoquat-methylsulfate |
| B-152 | one individualized compound I | Fenoxanil |
| B-153 | one individualized compound I | Folpet |
| B-154 | one individualized compound I | Oxolinsaure |
| B-155 | one individualized compound I | Piperalin |
| B-156 | one individualized compound I | Proquinazid |
| B-157 | one individualized compound I | Pyroquilon |
| B-158 | one individualized compound I | Quinoxyfen |
| B-159 | one individualized compound I | Triazoxid |
| B-160 | one individualized compound I | Tricyclazole |
| B-161 | one individualized compound I | 2-Butoxy-6-iodo-3-propyl-chromen-4- |
| | | one |
| B-162 | one individualized compound I | 5-Chloro-1-(4,6-dimethoxy-pyrimidin-2- |
| | | yl)-2-methyl-1H-benzoimidazole |
| B-163 | one individualized compound I | 5-Chloro-7-(4-methyl-piperidin-1-yl)- |
| | | 6-(2,4,6-trifluoro-phenyl)-[1,2,4]tri- |
| | | azolo[1,5-a]pyrimidine |
| B-164 | one individualized compound I | 5-ethyl-6-octy-[1,2,4]triazolo[1,5-a]pyrimidine- |
| | | 7-ylamine |
| B-165 | one individualized compound I | Ferbam |
| B-166 | one individualized compound I | Mancozeb |
| B-167 | one individualized compound I | Maneb |
| B-168 | one individualized compound I | Metam |
| B-169 | one individualized compound I | Methasulphocarb |
| B-170 | one individualized compound I | Metiram |
| B-171 | one individualized compound I | Propineb |
| B-172 | one individualized compound I | Thiram |
| B-173 | one individualized compound I | Zineb |
| B-174 | one individualized compound I | Ziram |
| B-175 | one individualized compound I | Diethofencarb |
| B-176 | one individualized compound I | Benthiavalicarb |
| B-177 | one individualized compound I | Iprovalicarb |
| B-178 | one individualized compound I | Propamocarb |
| B-179 | one individualized compound I | Propamocarb hydrochlorid |
| B-180 | one individualized compound I | Valiphenal |
| B-181 | one individualized compound I | N-(1-(1-(4- |
| | | cyanophenyl)ethanesulfonyl)-but-2-yl)carbamic |
| | | acid-(4-fluorophenyl) ester |
| B-182 | one individualized compound I | Dodine |
| B-183 | one individualized compound I | Dodine free base |
| B-184 | one individualized compound I | Guazatine |
| B-185 | one individualized compound I | Guazatine-acetate |
| B-186 | one individualized compound I | Iminoctadine |
| B-187 | one individualized compound I | Iminoctadine-triacetate |
| B-188 | one individualized compound I | Iminoctadine-tris(albesilate) |
| B-189 | one individualized compound I | Kasugamycin |
| B-190 | one individualized compound I | Kasugamycin-hydrochloride-hydrate |
| B-191 | one individualized compound I | Polyoxine |
| B-192 | one individualized compound I | Streptomycin |
| B-193 | one individualized compound I | Validamycin A |
| B-194 | one individualized compound I | Binapacryl |
| B-195 | one individualized compound I | Dicloran |
| B-196 | one individualized compound I | Dinobuton |
| B-197 | one individualized compound I | Dinocap |
| B-198 | one individualized compound I | Nitrothal-isopropyl |
| B-199 | one individualized compound I | Tecnazen |
| B-200 | one individualized compound I | Fentin salts |
| B-201 | one individualized compound I | Dithianon |
| B-202 | one individualized compound I | Isoprothiolane |
| B-203 | one individualized compound I | Edifenphos |
| B-204 | one individualized compound I | Fosetyl, Fosetyl-aluminium |
| B-205 | one individualized compound I | Iprobenfos |
| B-206 | one individualized compound I | Phosphorous acid (H₃PO₃) and derivatives |
| B-207 | one individualized compound I | Pyrazophos |
| B-208 | one individualized compound I | Tolclofos-methyl |
| B-209 | one individualized compound I | Chlorothalonil |
| B-210 | one individualized compound I | Dichlofluanid |
| B-211 | one individualized compound I | Dichlorophen |
| B-212 | one individualized compound I | Flusulfamide |
| B-213 | one individualized compound I | Hexachlorbenzene |
| B-214 | one individualized compound I | Pencycuron |
| B-215 | one individualized compound I | Pentachlorophenol and salts |
| B-216 | one individualized compound I | Phthalide |
| B-217 | one individualized compound I | Quintozene |
| B-218 | one individualized compound I | Thiophanate Methyl |
| B-219 | one individualized compound I | Tolylfluanid |
| B-220 | one individualized compound I | N-(4-chloro-2-nitro-phenyl)-N-ethyl- |
| | | 4-methyl-benzenesulfonamide |
| B-221 | one individualized compound I | Bordeaux mixture |
| B-222 | one individualized compound I | Copper acetate |
| B-223 | one individualized compound I | Copper hydroxide |
| B-224 | one individualized compound I | Copper oxychloride |
| B-225 | one individualized compound I | basic Copper sulfate |
| B-226 | one individualized compound I | Sulfur |
| B-227 | one individualized compound I | Biphenyl |
| B-228 | one individualized compound I | Bronopol |
| B-229 | one individualized compound I | Cyflufenamid |
| B-230 | one individualized compound I | Cymoxanil |
| B-231 | one individualized compound I | Diphenylamin |
| B-232 | one individualized compound I | Metrafenone |
| B-233 | one individualized compound I | Mildiomycin |
| B-234 | one individualized compound I | Oxin-copper |
| B-235 | one individualized compound I | Prohexadione calcium |
| B-236 | one individualized compound I | Spiroxamine |
| B-237 | one individualized compound I | Tolylfluanid |
| B-238 | one individualized compound I | N-(Cyclopropylmethoxyimino-(6- |
| | | difluoromethoxy-2,3-difluoro-phenyl)- |
| | | methyl)-2-phenyl acetamide |
| B-239 | one individualized compound I | N′-(4-(4-chloro-3-trifluoromethyl- |
| | | phenoxy)-2,5-dimethyl-phenyl)-N-ethyl- |
| | | N-methyl formamidine |
| B-240 | one individualized compound I | N′-(4-(4-fluoro-3-trifluoromethyl- |
| | | phenoxy)-2,5-dimethyl-phenyl)-N-ethyl- |
| | | N-methyl formamidine |
| B-241 | one individualized compound I | N′-(2-methyl-5-trifluoromethyl-4-(3-tri- |
| | | methylsilanyl-propoxy)-phenyl)-N-ethyl- |
| | | N-methyl formamidine |
| B-242 | one individualized compound I | N′-(5-difluoromethyl-2-methyl-4-(3-tri- |
| | | methylsilanyl-propoxy)-phenyl)-N-ethyl- |
| | | N-methyl formamidine |
| B-243 | one individualized compound I | 2-{1-[2-(5-Methyl-3-trifluoromethyl- |
| | | pyrazole-1-yl)-acetyl]-piperidin-4-yl}- |
| | | thiazole-4-carboxylic acid methyl- |
| | | (1,2,3,4-tetrahydro-naphthalen-1-yl)- |
| | | amide |
| B-244 | one individualized compound I | 2-{1-[2-(5-Methyl-3-trifluoromethyl- |
| | | pyrazole-1-yl)-acetyl]-piperidin-4-yl}- |
| | | thiazole-4-carboxylic acid methyl-(R)- |
| | | 1,2,3,4-tetrahydro-naphthalen-1-yl- |
| | | amide |
| B-245 | one individualized compound I | Acetic acid 6-tert.-butyl-8-fluoro-2,3- |
| | | dimethyl-quinolin-4-yl ester |
| B-246 | one individualized compound I | Methoxy-acetic acid 6-tert-butyl-8- |
| | | fluoro-2,3-dimethyl-quinolin-4-yl ester |
| B-247 | one individualized compound I | Carbaryl |
| B-248 | one individualized compound I | Carbofuran |
| B-249 | one individualized compound I | Carbosulfan |
| B-250 | one individualized compound I | Methomylthiodicarb |
| B-251 | one individualized compound I | Bifenthrin |
| B-252 | one individualized compound I | Cyfluthrin |
| B-253 | one individualized compound I | Cypermethrin |
| B-254 | one individualized compound I | alpha-Cypermethrin |
| B-255 | one individualized compound I | zeta-Cypermethrin |
| B-256 | one individualized compound I | Deltamethrin |
| B-257 | one individualized compound I | Esfenvalerate |
| B-258 | one individualized compound I | Lambda-cyhalothrin |
| B-259 | one individualized compound I | Permethrin |
| B-260 | one individualized compound I | Tefluthrin |
| B-261 | one individualized compound I | Diflubenzuron |
| B-262 | one individualized compound I | Flufenoxuron |
| B-263 | one individualized compound I | Lufenuron |
| B-264 | one individualized compound I | Teflubenzuron |
| B-265 | one individualized compound I | Spirotetramate |
| B-266 | one individualized compound I | Clothianidin |
| B-267 | one individualized compound I | Dinotefuran |
| B-268 | one individualized compound I | Imidacloprid |
| B-269 | one individualized compound I | Thiamethoxam |
| B-270 | one individualized compound I | Acetamiprid |
| B-271 | one individualized compound I | Thiacloprid |
| B-272 | one individualized compound I | Endosulfan |
| B-273 | one individualized compound I | Fipronil |
| B-274 | one individualized compound I | Abamectin |
| B-275 | one individualized compound I | Emamectin |
| B-276 | one individualized compound I | Spinosad |
| B-277 | one individualized compound I | Spinetoram |
| B-278 | one individualized compound I | Hydramethylnon |
| B-279 | one individualized compound I | Chlorfenapyr |
| B-280 | one individualized compound I | Fenbutatin oxide |
| B-281 | one individualized compound I | Indoxacarb |
| B-282 | one individualized compound I | Metaflumizone |
| B-283 | one individualized compound I | Flonicamid |
| B-284 | one individualized compound I | Lubendiamide |
| B-285 | one individualized compound I | Chlorantraniliprole |
| B-286 | one individualized compound I | Cyazypyr (HGW86) |
| B-287 | one individualized compound I | Cyflumetofen |
| B-288 | one individualized compound I | Acetochlor |
| B-289 | one individualized compound I | Dimethenamid |
| B-290 | one individualized compound I | metolachlor |
| B-291 | one individualized compound I | Metazachlor |
| B-292 | one individualized compound I | Glyphosate |
| B-293 | one individualized compound I | Glufosinate |
| B-294 | one individualized compound I | Sulfosate |
| B-295 | one individualized compound I | Clodinafop |
| B-296 | one individualized compound I | Fenoxaprop |
| B-297 | one individualized compound I | Fluazifop |
| B-298 | one individualized compound I | Haloxyfop |
| B-299 | one individualized compound I | Paraquat |
| B-300 | one individualized compound I | Phenmedipham |
| B-301 | one individualized compound I | Clethodim |
| B-302 | one individualized compound I | Cycloxydim |
| B-303 | one individualized compound I | Profoxydim |
| B-304 | one individualized compound I | Sethoxydim |
| B-305 | one individualized compound I | Tepraloxydim |
| B-306 | one individualized compound I | Pendimethalin |
| B-307 | one individualized compound I | Prodiamine |
| B-308 | one individualized compound I | Trifluralin |
| B-309 | one individualized compound I | Acifluorfen |
| B-310 | one individualized compound I | Bromoxynil |
| B-311 | one individualized compound I | Imazamethabenz |
| B-312 | one individualized compound I | Imazamox |
| B-313 | one individualized compound I | Imazapic |
| B-314 | one individualized compound I | Imazapyr |
| B-315 | one individualized compound I | Imazaquin |
| B-316 | one individualized compound I | Imazethapyr |
| B-317 | one individualized compound I | 2,4-Dichlorophenoxyacetic acid (2,4-D) |
| B-318 | one individualized compound I | Chloridazon |
| B-319 | one individualized compound I | Clopyralid |
| B-320 | one individualized compound I | Fluroxypyr |
| B-321 | one individualized compound I | Picloram |
| B-322 | one individualized compound I | Picolinafen |
| B-323 | one individualized compound I | Bensulfuron |
| B-324 | one individualized compound I | Chlorimuron-ethyl |
| B-325 | one individualized compound I | Cyclosulfamuron |
| B-326 | one individualized compound I | Iodosulfuron |
| B-327 | one individualized compound I | Mesosulfuron |
| B-328 | one individualized compound I | Metsulfuron-methyl |
| B-329 | one individualized compound I | Nicosulfuron |
| B-330 | one individualized compound I | Rimsulfuron |
| B-331 | one individualized compound I | Triflusulfuron |
| B-332 | one individualized compound I | Atrazine |
| B-333 | one individualized compound I | Hexazinone |
| B-334 | one individualized compound I | Diuron |
| B-335 | one individualized compound I | Florasulam |
| B-336 | one individualized compound I | Pyroxasulfone |
| B-337 | one individualized compound I | Bentazone |
| B-338 | one individualized compound I | Cinidon-ethlyl |
| B-339 | one individualized compound I | Cinmethylin |
| B-340 | one individualized compound I | Dicamba |
| B-341 | one individualized compound I | Diflufenzopyr |
| B-342 | one individualized compound I | Quinclorac |
| B-343 | one individualized compound I | Quinmerac |
| B-344 | one individualized compound I | Mesotrione |
| B-345 | one individualized compound I | Saflufenacil |
| B-346 | one individualized compound I | Topramezone |

The active substances referred to as component 2, their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624).

The mixtures of active substances can be prepared as compositions comprising besides the active ingredients at least one inert ingredient by usual means, e.g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effectiveness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

### I. SYNTHESIS EXAMPLES

With due modification of the starting compounds, the procedures shown in the synthesis examples below were used to obtain further compounds I. The resulting compounds, together with physical data, are listed in Table I below.

HPC/MS conditions were as follows: HPLC column: RP-18 column (Chromolith Speed ROD from Merck KgaA, Germany), 50 mm×4.6 mm; Eluent: acetonitrile+0.1% trifluoroacetic acid (TFA)/water+0.1% TFA (gradient from 5:95 to 95:5 in 5 min at 40° C., flow of 1.8 ml/min; MS: Quadrupol Elektrospray Ionisation, 80 V (positive mode).

### Example 1

### Preparation of 2-(1-methyl-5-trifluoromethyl-1H-pyrazol-3-yloxy)-thiazole-4-sulfonic acid (2-methoxy-pyridin-4-ylmethyl)-amide (Table I, example I-1)

### Example 1.1

### Preparation of 3-(thiazol-2-yloxy)-pyridine

Pyridin-3-ol (8.7 g) in dry dimethylacetamide (150 ml) under nitrogen was treated with sodium hydride (2.54 g). After the initial effervescence ceased 2-bromothiazol (15 g) and Kl (13.6 g) were added. The mixture was stirred for 23 h at 140° C. and the resulting reaction mixture was partitioned between MTBE and water. The aqueous layer was extracted with MTBE and the combined organic layers were washed with brine solution and the solvent removed from the organic layers under reduced pressure. 3-(Thiazol-2-yloxy)-pyridine was isolated as a red oil (2.95 g). ¹H-NMR (CDCl₃): δ=6.95 (s, 1H), 7.2 (s, 1H), 7.38 (m, 1H), 7.8 (m, 1H), 8.5 (m, 1H), 8.65 ppm (s, 1H).

### Example 1.2

### Preparation of 2-(pyridin-3-yloxy)-thiazole-5-sulfonyl chloride

A stirred solution of 3-(thiazol-2-yloxy)-pyridine (1.0 g) in anhydrous THF (50 ml) was treated dropwise at −78° C. with tert.-butyllithium (4.7 ml) and stirred for 1 hour at −70° C. Sulphur dioxide (12.5 g) was bubbled through the reaction mixture for about 10 min and after 30 min at −70° C. the mixture was stirred at 23° C. for 15 min. The solvents were removed by rotary evaporation and the residue was dissolved in 50 ml DCM and N-chlorosuccinimide (1.0 g) was added. After stirring at about 20° C. for 1 hour the reaction mixture was poured into 120 ml of 3 N hydrochloric acid solution and extracted with dichloromethane. The combined organic phases were washed once with saturated brine and after removal of the solvent under reduced pressure the crude subtitle product (1.65 g as brownish oil) was obtained and used without further purification.

### Example 1.3

### Preparation of 2-(pyridin-3-yloxy)-thiazole-5-sulfonic acid-(2,3-dimethyl-pyridin-4-ylmethyl)-amide (Table I, example I-1)

To a solution of 4-(aminomethyl)-2,3-dimethylpyridine (0.150 g) in DCM (5 ml) was added N,N-diisopropylethylamine (0.356 g, 2.75 mmol). The reaction mixture was cooled to 0° C., 2-(pyridin-3-yloxy)-thiazole-5-sulfonyl chloride (0.3 g, example 1.2) was added and the reaction mixture was stirred 72 h at 23° C. Subsequently, the solvent was removed in vacuum. The residue obtained was purified by MPL-chromatography (CH₃CN/water) to yield the title compound (80 mg). ¹H-NMR (CDCl₃): δ=2.25 (s, 3H), 2.5 (s, 3H), 4.3 (s, 2H), 5.9 (s, 1H), 7.05 (m, 1H), 7.5 ppm (m, 1H).

### Example 2

### Preparation of 2-(1-methyl-5-trifluoromethyl-1H-pyrazol-3-yloxy)-thiazole-5-sulfonic acid (2-methoxy-pyridin-4-ylmethyl)-amide (Table I, example 1-4)

### Example 2.1

### Preparation of 2-(1-methyl-5-trifluoromethyl-1H-pyrazol-3-yloxy)-thiazole

1-Methyl-5-trifluoromethyl-1H-pyrazol-3-ol (5.1 g) in dry DMF (50 ml) under nitrogen was treated at 0° C. with sodium hydride (0.82 g). After the initial effervescence ceased 2-bromothiazol (5 g) and Kl (4.4 g) were added. The mixture was stirred for 23 h at 140° C., the solvent was removed by rotary evaporation and the residue was purified by flash column chromatography on silica gel (cyclohexan/ethyl acetate) to yield the title compound as yellow oil (4.94 g). ¹H-NMR (CDCl₃): δ=3.98 (s, 3H), 6.6 (s, 1H), 6.9 (s, 1H), 7.3 ppm (s, 1H)

### Example 2.2

### Preparation of 2-(1-methyl-5-trifluoromethyl-1H-pyrazol-3-yloxy)-thiazole-5-sulfonyl chloride

A stirred solution of 2-(1-methyl-5-trifluoromethyl-1H-pyrazol-3-yloxy)-thiazole (0.5 g) in anhydrous THF (22 ml) was treated dropwise at −70° C. with tert.-butyllithium (1.7 ml) and stirred for 1 hour at −70° C. Sulphur dioxide (12.5 g, 196.4 mmol) was bubbled through the reaction mixture at −30° C. for about 10 min and after 1 hour at −30° C. the mixture was stirred at 23° C. for 1 hour. The solvents were removed by rotary evaporation and the residue was dissolved in 30 ml DCM and N-chlorosuccinimide (0.38 g) was added. After stirring at room temperature for 1 hour the suspension was filtered through a bed of Celite, the organic phase was concentrated under reduced pressure and the crude subtitle product was obtained as yellow oil (0.8 g) and used in the next step without further purification.

### Example 2.3

### Preparation of 2-(1-methyl-5-trifluoromethyl-1H-pyrazol-3-yloxy)-thiazole-5-sulfonic acid (2-methoxy-pyridin-4-ylmethyl)-amide (Table I, example I-4)

To a solution of 4-(aminomethyl)-2-methoxypyridine (0.15 g) in DCM (5 ml) was added N,N-diisopropylethylamine (0.35 g). The reaction mixture was cooled to 0° C., 2-(1-methyl-5-trifluoromethyl-1H-pyrazol-3-yloxy)-thiazole-4-sulfonyl chloride (0.37 g, example 2.2) was added and the reaction mixture was stirred 30 h at 23° C. Subsequently the solvent was removed in vacuum. The residue obtained was purified by flash column chromatography on silica gel (cyclohexan/ethyl acetate, 1:1) afforded the title compound (70 mg). ¹H-NMR (CDCl₃): δ=3.8 (s, 3H), 3.9 (s, 3H), 4.2 (s, 2H), 6.5 (s, 1H), 6.65 (s, 1H), 6.8 (m, 1H), 7.7 (s, 1H) and 8.1 ppm (m, 1H).

TABLE I
Compounds of formula I.

| | | | | m.p. [° C.]; |
|---|---|---|---|---|
| | | | | HPLC Rₜ [min], |
| No. | (R^{a})ₙ | R | A-Y-Het | MS (M + H⁺) |
| I-1 | 2,3- | H | 2-(pyridyl-3-oxy)-thiazol-5-yl | 1.62 min, 376.70 |
| | (CH₃)₂ | | | |
| I-2 | 2-OCH₃ | H | 2-(pyridyl-3-oxy)-thiazol-5-yl | 1.87 min, 378.70 |
| I-3 | 2,3- | H | 2-(1-methyl-5-trifluoromethyl- | 2.54 min, 447.60 |
| | (CH₃)₂ | | 1H-pyrazol-3-yloxy)-thiazol-5-yl | |
| I-4 | 2-OCH₃ | H | 2-(1-methyl-5-trifluoromethyl- | 2.83 min, 449.60 |
| | | | 1H-pyrazol-3-yloxy)-thiazol-5-yl | |
| I-5 | 2,3- | H | 6-(2-methyl-5-trifluoromethyl- | 161° C. |
| | (CH₃)₂ | | 2H-pyrazol-3-yloxy)-pyridin- | |
| | | | 2-yl | |

### II. EXAMPLES OF THE ACTION AGAINST HARMFUL FUNGI

### II.A Glasshouse Trials

The active compounds were formulated separately or together as a stock solution comprising 25 mg of active compound which was made up to 10 ml using a mixture of acetone and/or dimethyl sulfoxide (DMSO) and the emulsifier Uniperol® EL (wetting agent having emulsifying and dispersing action based on ethoxylated alkylphenols) in a volume ratio of solvent/emulsifier of 99:1. This solution was then made up to 100 ml using water. This stock solution was diluted with the solvent/emulsifier/water mixture described to the active compound concentration given below.

### Use Example 1

### Protective Action Against Early Blight on Tomatoes Caused by Phytophthora infestans

Young seedlings of tomato plants were grown in pots. The plants were sprayed to runoff with an aqueous suspension containing the concentration of active ingredient stated below. The next day, the treated plants were inoculated with an aqueous suspension of sporangia of Phytophthora infestans. After inoculation, the trial plants were immediately transferred to a humid chamber. After 6 days at 18 to 20° C. and a relative humidity close to 100%, the extent of fungal attack on the leaves was visually assessed as % diseased leaf area.

1-15. (canceled)

16. A compound of formula I 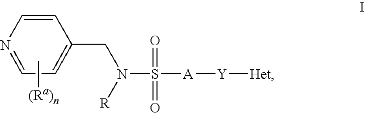
wherein:
R^{a} is halogen, CN, NH₂, NO₂, OH, SH, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylamino, di(C₁-C₄-alkyl)amino, C₂-C₄-alkenyl, C₂-C₄-haloalkenyl, C₂-C₄-alkynyl, C₂-C₄-haloalkynyl, C₁-C₄-alkoxy-C₁-C₄-alkyl, C₂-C₄-alkenyl, C₂-C₄-alkynyl, C₃-C₈-cycloalkyl or C₁-C₄-alkyl-C₃-C₈-cycloalkyl; and/or
two radicals R^{a} that are bound to adjacent ring member atoms of the pyridine ring may form together with said ring member atoms a fused 5-, 6- or 7-membered saturated, partially unsaturated or aromatic cycle, which may be a carbocycle or heterocycle, wherein the ring member atoms of the fused heterocycle include, besides carbon atoms, 1, 2, 3 or 4 heteroatoms selected from the group consisting of N, O and S, and wherein the fused cycle is unsubstituted or carries 1, 2, 3 or 4 identical or different groups as defined for R^{a};
n indicates the number of substituents R^{a} on the pyridine ring and n is 0, 1, 2, 3 or 4, wherein R^{a} radicals are identical or different if n is 2, 3 or 4;
R is hydrogen, C₁-C₄-alkyl, C₁-C₄-haloalkyl, C₂-C₄-alkenyl, C₂-C₄-alkynyl, C₃-C₈-cycloalkyl, C₁-C₄-alkyl-C₃-C₈-cycloalkyl or benzyl wherein the phenyl moiety of benzyl is unsubstituted or carries 1, 2, 3, 4 or 5 substituents selected from the group consisting of cyano, halogen, C₁-C₄-alkyl, C₁-C₄-haloalkyl, C₁-C₄-alkoxy, C₁-C₄-haloalkoxy, C₁-C₄-alkoxycarbonyl and di(C₁-C₄-alkyl)aminocarbonyl;
A is a 5- or 6-membered heteroarenediyl, wherein the ring member atoms of the heteroaryl include, besides carbon atoms, 1, 2, 3 or 4 heteroatoms selected from the group consisting of N, O and S, and wherein the heteroaryl is unsubstituted or carries 1, 2, 3 or 4 identical or different groups R^{b}:
R^{b} is halogen, CN, NO₂, C₁-C₄-alkyl, C₁-C₄-haloalkyl, C₁-C₄-alkoxy, C₁-C₄-haloalkoxy, C₂-C₄-alkenyl, C₂-C₄-haloalkenyl, C₂-C₄-alkynyl, C₂-C₄-haloalkynyl, C₁-C₄-alkylcarbonyl, C₁-C₄-alkoxycarbonyl, C₁-C₄-alkylamino, di(C₁-C₄-alkyl)amino, C₁-C₄-alkylaminocarbonyl or di(C₁-C₄-alkyl)aminocarbonyl;
Y is a divalent group selected from —O—, —O—CH₂—, —CH₂—O—, —S—, —S(═O)—, —S(═O)₂—, C₁-C₄-alkanediyl, —N(R^{Π})— or —C(NOR^{Π})—,
R^{Π} is hydrogen or C₁-C₄-alkyl:
Het is a pyridyl radical selected from the group consisting of pyridin-2-yl, pyridin-3-yl and pyridin-4-yl, wherein the aforementioned pyridyl radicals are unsubstituted or carry 1, 2, 3 or 4 identical or different substituents R^{c},
a pyridazinyl radical selected from the group consisting of pyridazin-3-yl and pyridazin-4-yl, wherein the aforementioned pyridazinyl radicals are unsubstituted or carry 1, 2 or 3 identical or different substituents R^{c},
a pyrimidinyl radical selected from the group consisting of pyrimidin-2-yl, pyrimidin-4-yl, pyrimidin-5-yl and pyrimidin-6-yl, wherein the aforementioned pyrimidinyl radicals are unsubstituted or carry 1, 2 or 3 identical or different substituents R^{c},
a pyrazinyl radical selected from the group consisting of pyrazin-2-yl and pyrazin-3-yl, wherein the aforementioned pyrazinyl radicals are unsubstituted or carry 1, 2 or 3 identical or different substituents R^{c},
a triazinyl radical selected from the group consisting of 1,2,4-triazin-2-yl and 1,2,4-triazin-3-yl, wherein the aforementioned pyrazinyl radicals are unsubstituted or carry 1, 2 or 3 identical or different substituents R^{c},
a pyrazolyl radical selected from the group consisting of pyrazol-3-yl, pyrazol-4-yl and pyrazol-5-yl, wherein the aforementioned pyrazolyl radicals are unsubstituted or carry 1, 2 or 3 identical or different substituents R^{c}, or
an isoxazolyl radical selected from the group consisting of isoxazol-3-yl, isoxazol-4-yl and isoxazol-5-yl, wherein the aforementioned isoxazolyl radicals are unsubstituted or carry one or two identical or different substituents:
R^{c} is halogen, CN, NO₂, NH₂, C₁-C₆-alkyl, C₁-C₆-haloalkyl, C₁-C₆-alkoxy, C₁-C₆-haloalkoxy, C₁-C₆-alkylamino, di(C₁-C₆-alkyl)amino, C₁-C₆-alkyl-thio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₁-C₆-alkoxy-C₁-C₄-alkyl, C₁-C₆-haloalkoxy-C₁-C₄-alkyl, C₂-C₆-alkenyl, C₂-C₆-alkynyl, C(═O)R′, C(═NOR″)R′″, C₃-C₈-cycloalkyl, C₁-C₄-alkyl-C₃-C₈-cycloalkyl, phenyl, phenoxy, phenoxy-C₁-C₄-alkyl or a 5- or 6-membered heteroaryl, wherein the ring member atoms of the heteroaryl include, besides carbon atoms, 1, 2, 3 or 4 heteroatoms selected from the group consisting of N, O and S, and wherein the aforementioned cyclic radicals are unsubstituted or carry 1, 2, 3 or 4 identical or different substituents R^{d}:
R′ is hydrogen, NH₂, C₁-C₄-alkyl, C₁-C₄-haloalkyl, C₂-C₄-alkenyl, C₂-C₄-alkynyl, C₁-C₄-alkoxy, C₁-C₄-alkoxy-C₁-C₄-alkoxy, C₁-C₄-haloalkoxy, C₁-C₄-alkylamino or di(C₁-C₄-alkyl)amino;
R″ is hydrogen, C₁-C₄-alkyl, C₁-C₄-haloalkyl, C₂-C₄-alkenyl, C₂-C₄-alkynyl or C₁-C₄-alkoxy-C₁-C₄-alkyl,
R′″ is hydrogen or C₁-C₄-alkyl;
R^{d} is halogen, CN, C₁-C₄-alkyl, C₁-C₄-haloalkyl, C₁-C₄-alkoxy or C₁-C₄-haloalkoxy;
and/or two radicals R^{c} that are bound to adjacent ring member atoms of the Het group may form together with said ring member atoms a fused 5-, 6- or 7-membered saturated, partially unsaturated or aromatic cycle, which may be a carbocycle or heterocycle, wherein the ring member atoms of the fused heterocycle include, besides carbon atoms, 1, 2, 3 or 4 heteroatoms selected from the group consisting of N, O and S, and wherein the fused cycle is unsubstituted or carries 1, 2, 3 or 4 identical or different radicals R^{e}:
R^{e} is halogen, CN, C₁-C₄-alkyl, C₁-C₄-haloalkyl, C₁-C₄-alkoxy or C₁-C₄-haloalkoxy;
and/or the N-oxide and/or the agriculturally acceptable salt thereof.

17. The compound according to claim 16, wherein R is hydrogen.

18. The compound according to claim 16, wherein Y is —O—, —S— or —NH—.

19. The compound according to claim 18, wherein Y is —O—.

20. The compound according to claim 16, wherein A is thiendiyl, furandiyl or pyridindiyl.

21. The compound according to claims 16, wherein Het is pyridin-2-yl, which is unsubstituted or carries 1 or 2 radicals R^{c}.

22. The compound according to claim 21, wherein Het is selected from the group consisting of 3-trifluoromethylpyridin-2-yl, 5-trifluoromethylpyridin-2-yl, 4-trifluoromethylpyridin-2-yl, 3-chloropyridin-2-yl, 5-chloropyridin-2-yl, 4-chloropyridin-2-yl, 3-bromopyridin-2-yl, 5-bromopyridin-2-yl, 4-bromopyridin-2-yl, 3-trichloromethylpyridin-2-yl, 5-trichloromethylpyridin-2-yl, 4-trichloromethylpyridin-2-yl, 3-cyanopyridin-2-yl, 5-cyanopyridin-2-yl, 4-cyanopyridin-2-yl, 3-nitropyridin-2-yl, 5-nitropyridin-2-yl, 4-nitropyridin-2-yl, 3-methylsulfonylpyridin-2-yl, 5-methylsulfonylpyridin-2-yl, 4-methylsulfonylpyridin-2-yl, 3-ethyl sulfonylpyridin-2-yl, 5-ethylsulfonylpyridin-2-yl, 4-ethylsulfonylpyridin-2-yl, 3-methoxycarbonylpyridin-2-yl, 5-methoxycarbonylpyridin-2-yl, 4-methoxycarbonylpyridin-2-yl, 5-aminocarbonylpyridin-2-yl, 4-aminocarbonylpyridin-2-yl, 3-aminocarbonylpyridin-2-yl, 5-N-methylaminocarbonylpyridin-2-yl, 4-N-methylaminocarbonylpyridin-2-yl, 3-N-methylaminocarbonylpyridin-2-yl, 3-methoxypyridin-2-yl, 3-ethoxypyridin-2-yl, 3-difluoromethoxypyridin-2-yl, 5-methoxypyridin-2-yl, 5-ethoxypyridin-2-yl, 5-difluoromethoxypyridin-2-yl, 3-chloro-5-trifluoromethylpyridin-2-yl, 3-fluoro-5-trifluoromethylpyridin-2-yl, 3-bromo-5-trifluoromethylpyridin-2-yl, 3-methyl-5-trifluoromethylpyridin-2-yl, 3-ethyl-5-trifluoromethylpyridin-2-yl, 3-chloro-5-difluoromethoxypyridin-2-yl, 3-fluoro-5-difluoromethoxypyridin-2-yl, 3-methyl-5-difluoromethoxypyridin-2-yl, 3-chloro-5-trichloromethylpyridin-2-yl, 3-fluoro-5-trichloromethylpyridin-2-yl, 3-chloro-5-cyanopyridin-2-yl, 3-fluoro-5-cyanopyridin-2-yl, 3-methyl-5-cyanopyridin-2-yl, 3-ethyl-5-cyanopyridin-2-yl, 3-chloro-5-nitropyridin-2-yl, 3-chloro-5-methoxycarbonylpyridin-2-yl, 3-chloro-5-aminocarbonylpyridin-2-yl, 3-chloro-5-methylaminocarbonylpyridin-2-yl, 3-fluoro-5-nitropyridin-2-yl, 3-fluoro-5-methoxycarbonylpyridin-2-yl, 3-fluoro-5-aminocarbonylpyridin-2-yl, 3-fluoro-5-methylaminocarbonylpyridin-2-yl, 4-chloro-5-trifluoromethylpyridin-2-yl, 4-fluoro-5-trifluoromethylpyridin-2-yl, 4-bromo-5-trifluoromethylpyridin-2-yl, 4-methyl-5-trifluoromethylpyridin-2-yl, 4-chloro-5-nitropyridin-2-yl, 4-chloro-5-cyanopyridin-2-yl, 3-chloro-6-trifluoromethylpyridin-2-yl, 3-fluoro-6-trifluoromethylpyridin-2-yl, 3-methyl-6-trifluoromethylpyridin-2-yl, 4-chloro-5-difluoromethoxypyridin-2-yl, 4-fluoro-5-difluoromethoxypyridin-2-yl, 3-chloro-5-bromopyridin-2-yl, 3,5-dichloropyridin-2-yl, 3,5-difluoropyridin-2-yl, 3,5-dibromopyridin-2-yl, 3-methyl-5-chloropyridin-2-yl, 3-methyl-5-fluoropyridin-2-yl, 3-methyl-5-bromopyridin-2-yl, 3-methoxy-5-trifluoromethylpyridin-2-yl, 3-methoxy-5-cyanopyridin-2-yl, 3-methoxy-5-nitropyridin-2-yl, 3-methoxy-5-difluoromethoxypyridin-2-yl, 3-ethoxy-5-trifluoromethylpyridin-2-yl, 3-ethoxy-5-cyanopyridin-2-yl, 3-ethoxy-5-nitropyridin-2-yl, 3-ethoxy-5-difluoromethoxypyridin-2-yl, 3-chloro-4-methyl-5-trifluoromethylpyridin-2-yl and 3,4-dichloro-5-trifluoromethylpyridin-2-yl.

23. The compound according to claim 16, wherein Het carries 1, 2, 3 or 4 radicals R^{c} which are selected from the group consisting of F, Cl, Br, CN, C₁-C₂-alkylsulfonyl, C₁-C₂-alkoxycarbonyl, aminocarbonyl, C₁-C₂-alkylaminocarbonyl, di(C₁-C₂-alkyl)-aminocarbonyl, C₁-C₂-alkoxy, CF₃, CHF₂, OCF₃ and OCHF₂.

24. A process for preparing compounds I according to claim 16, which comprises reacting aminomethylpyridine compounds of formula II 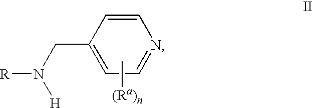
under basic conditions with a sulfonic acid derivative of formula III 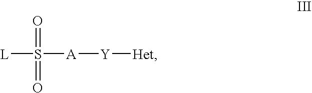
wherein the leaving group L is hydroxy or halogen.

25. An agrochemical composition comprising a solvent or solid carrier and at least a compound of claim 16, or an N-oxide or an agriculturally acceptable salt thereof.

26. The composition according to claim 25 comprising at least one further active substance.

27. A method for combating phytopathogenic harmful fungi, which process comprises treating the fungi or the materials, plants, the soil or seeds to be protected against fungal attack, with an effective amount of at least one compound of claim 16, or an N-oxide or an agriculturally acceptable salt thereof.

28. The method according to claim 27, wherein R is hydrogen.

29. The method according to claim 27, wherein Y is —O—, —S— or —NH—.

30. The method according to claim 29, wherein Y is —O—.

31. The method according to claim 27, wherein A is thiendiyl, furandiyl or pyridindiyl.

32. The method according to claim 27, wherein Het is pyridin-2-yl, which is unsubstituted or carries 1 or 2 radicals R^{c}.

33. The method according to claim 32, wherein Het is selected from the group consisting of 3-trifluoromethylpyridin-2-yl, 5-trifluoromethylpyridin-2-yl, 4-trifluoromethylpyridin-2-yl, 3-chloropyridin-2-yl, 5-chloropyridin-2-yl, 4-chloropyridin-2-yl, 3-bromopyridin-2-yl, 5-bromopyridin-2-yl, 4-bromopyridin-2-yl, 3-trichloromethylpyridin-2-yl, 5-trichloromethylpyridin-2-yl, 4-trichloromethylpyridin-2-yl, 3-cyanopyridin-2-yl, 5-cyanopyridin-2-yl, 4-cyanopyridin-2-yl, 3-nitropyridin-2-yl, 5-nitropyridin-2-yl, 4-nitropyridin-2-yl, 3-methylsulfonylpyridin-2-yl, 5-methylsulfonylpyridin-2-yl, 4-methylsulfonylpyridin-2-yl, 3-ethylsulfonylpyridin-2-yl, 5-ethylsulfonylpyridin-2-yl, 4-ethylsulfonylpyridin-2-yl, 3-methoxycarbonylpyridin-2-yl, 5-methoxycarbonylpyridin-2-yl, 4-methoxycarbonylpyridin-2-yl, 5-aminocarbonylpyridin-2-yl, 4-aminocarbonylpyridin-2-yl, 3-aminocarbonylpyridin-2-yl, 5-N-methylaminocarbonylpyridin-2-yl, 4-N-methylaminocarbonylpyridin-2-yl, 3-N-methylaminocarbonylpyridin-2-yl, 3-methoxypyridin-2-yl, 3-ethoxypyridin-2-yl, 3-difluoromethoxypyridin-2-yl, 5-methoxypyridin-2-yl, 5-ethoxypyridin-2-yl, 5-difluoromethoxypyridin-2-yl, 3-chloro-5-trifluoromethylpyridin-2-yl, 3-fluoro-5-trifluoromethylpyridin-2-yl, 3-bromo-5-trifluoromethylpyridin-2-yl, 3-methyl-5-trifluoromethylpyridin-2-yl, 3-ethyl-5-trifluoromethylpyridin-2-yl, 3-chloro-5-difluoromethoxypyridin-2-yl, 3-fluoro-5-difluoromethoxypyridin-2-yl, 3-methyl-5-difluoromethoxypyridin-2-yl, 3-chloro-5-trichloromethylpyridin-2-yl, 3-fluoro-5-trichloromethylpyridin-2-yl, 3-chloro-5-cyanopyridin-2-yl, 3-fluoro-5-cyanopyridin-2-yl, 3-methyl-5-cyanopyridin-2-yl, 3-ethyl-5-cyanopyridin-2-yl, 3-chloro-5-nitropyridin-2-yl, 3-chloro-5-methoxycarbonylpyridin-2-yl, 3-chloro-5-aminocarbonylpyridin-2-yl, 3-chloro-5-methylaminocarbonylpyridin-2-yl, 3-fluoro-5-nitropyridin-2-yl, 3-fluoro-5-methoxycarbonylpyridin-2-yl, 3-fluoro-5-aminocarbonylpyridin-2-yl, 3-fluoro-5-methylaminocarbonylpyridin-2-yl, 4-chloro-5-trifluoromethylpyridin-2-yl, 4-fluoro-5-trifluoromethylpyridin-2-yl, 4-bromo-5-trifluoromethylpyridin-2-yl, 4-methyl-5-trifluoromethylpyridin-2-yl, 4-chloro-5-nitropyridin-2-yl, 4-chloro-5-cyanopyridin-2-yl, 3-chloro-6-trifluoromethylpyridin-2-yl, 3-fluoro-6-trifluoromethylpyridin-2-yl, 3-methyl-6-trifluoromethylpyridin-2-yl, 4-chloro-5-difluoromethoxypyridin-2-yl, 4-fluoro-5-difluoromethoxypyridin-2-yl, 3-chloro-5-bromopyridin-2-yl, 3,5-dichloropyridin-2-yl, 3,5-difluoropyridin-2-yl, 3,5-dibromopyridin-2-yl, 3-methyl-5-chloropyridin-2-yl, 3-methyl-5-fluoropyridin-2-yl, 3-methyl-5-bromopyridin-2-yl, 3-methoxy-5-trifluoromethylpyridin-2-yl, 3-methoxy-5-cyanopyridin-2-yl, 3-methoxy-5-nitropyridin-2-yl, 3-methoxy-5-difluoromethoxypyridin-2-yl, 3-ethoxy-5-trifluoromethylpyridin-2-yl, 3-ethoxy-5-cyanopyridin-2-yl, 3-ethoxy-5-nitropyridin-2-yl, 3-ethoxy-5-difluoromethoxypyridin-2-yl, 3-chloro-4-methyl-5-trifluoromethylpyridin-2-yl and 3,4-dichloro-5-trifluoromethylpyridin-2-yl.

34. The method according to claim 27, wherein Het carries 1, 2, 3 or 4 radicals R^{c} which are selected from the group consisting of F, Cl, Br, CN, C₁-C₂-alkylsulfonyl, C₁-C₂-alkoxycarbonyl, aminocarbonyl, C₁-C₂-alkylaminocarbonyl, di(C₁-C₂-alkyl)-aminocarbonyl, C₁-C₂-alkoxy, CF₃, CHF₂, OCF₃ and OCHF₂.

35. A seed comprising a compound of claim 16, or an N-oxide or an agriculturally acceptable salt thereof, in an amount of from 0.1 g to 10 kg per 100 kg of seed.
